(19) United States
(12) Patent Application Publication
(10) Pub. No.: US 2007/0275386 A1
(43) Pub. Date: Nov. 29, 2007

(54) Polymorphisms in the Epidermal Growth Factor Receptor Gene Promoter

(76) Inventors: Mark J. Ratain, Chicago, IL (US); Wanqing Liu, Chicago, IL (US); Federico Innocenti, Chicago, IL (US)

Correspondence Address: FULBRIGHT & JAWORSKI L.L.P., 600 CONGRESS AVE., SUITE 2400, AUSTIN, TX, 78701, US

(21) Appl. No.: 10/591,228

(22) PCT Filed: Mar. 1, 2005
(86) PCT No.: PCT/US05/06559

§ 371 Date: Jul. 18, 2007

Related U.S. Application Data
(60) Provisional application No. 60/549,069, filed on Mar. 1, 2004.

(51) Int. Cl. C12Q 1/68; G01N 33/53

(52) U.S. Cl. 435/6; 435/7.1

(57) ABSTRACT

The present invention concerns polymorphisms in the epidermal growth factor receptor (EGFR), gene. In some embodiments, the present invention is directed at compositions and methods involving single nucleotide polymorphisms (SNPs) in the promoter of the EGFR gene that affect EGFR expression. The identification of polymorphisms associated with EGFR expression or activity enables novel methods and compositions for evaluating the potential efficacy and toxicity of an EGFR-targeting therapeutic agent, predicting a patient's clinical prognosis, and evaluating a patient's risk of developing a disease that is associated with EGFR dysregulation.

The present invention claims priority to U.S. Provisional Patent Application Ser. No. 60/549,069, filed on Mar. 1, 2004, which is hereby incorporated by reference. The government owns rights in the present invention pursuant to grant number U01GM61393 from the National Institutes of Health.

### BACKGROUND OF THE INVENTION

1. Field of the Invention

The present invention relates generally to the field of molecular biology and oncology. More particularly, it concerns polymorphisms in the epidermal growth factor receptor (EGFR) gene associated with EGFR expression and activity. In some embodiments, the present invention is directed at compositions and methods involving single nucleotide polymorphisms (SNPs) in the promoter of the EGFR gene that affect EGFR expression.

2. Description of Related Art

Human epidermal growth factor receptor (EGFR) plays a critical role in the signal transduction pathway of cell proliferation, differentiation and survival. Overexpression of EGFR is found in about 30% of human primary tumors. Its activation in these tumors appears to promote tumor growth by increasing cell proliferation, motility, adhesion, invasive capacity, and by blocking apoptosis (Tysnes et al., 1997). EGFR overexpression and dysregulation has been associated with poorer prognosis in patients, and with metastasis, late-stage disease, and resistance to chemotherapy, hormonal therapy, and radiotherapy (Salomon et al., 1995; Akimoto et al., 1999; Wosikowski et al., 2000).

The EGFR 5′ regulatory region spans about 4 kb covering 2 kb upstream and 2 kb downstream of exon 1. The regulatory elements include a promoter region and two separate enhancer regions. The function of the EGFR promoter and enhancers are well studied and documented (Ishii et al., 1985; Haley et al., 1987; Johnson et al., 1988; Kageyama et al., 1988; Maekawa et al., 1989). Briefly, there is no TATA or CAAT box found in the promoter. Instead, there are multiple transcription initiation sites (Ishii et al., 1985; Haley et al., 1987; Johnson et al., 1988; Kageyama et al., 1988). A number of cis- and trans-regulators have been discovered. These regulators include EGF responsive DNA-binding protein (ERDBP-1), p53, p63, Sp1, Vitamin D-responsive element (VDRE) and estrogen responsive element, which reflects the perplexing regulation of EGFR.

Deoxyribonuclease I footprinting showed that Sp1 can bind to four CCGCCC sequences (−457 to −440, −365 to −286, −214 to −200, and −110 to −84) in the EGFR gene promoter and may, therefore, play a vital role in the gene regulation (Johnson et al., 1998). Studies by Gebhardt and colleagues (1999) demonstrated that a dinucleotide (CA)n repeat polymorphism in the intron 1 of EGFR (near the downstream enhancer) ranging from 14 to 21 repeats, appears to regulate EGFR expression. The longer allele with 21 repeats showed an 80% reduction of gene expression compared to the shorter allele with 16 repeats (Gebhardt et al., 1999; Buerger et al., 2000). Data from studies on the polymorphic CA repeat suggest that this polymorphic site may play a role in cancer susceptibility (Brandt et al., 2004).

Given the importance of EGFR in tumor biology, several EGFR-targeted cancer therapies are currently under development. EGFR-targeting agents are typically directed to inhibiting EGFR phosphorylation or blocking EGF binding. One drug that was recently approved for the treatment of metastatic non-small cell lung cancer is gefitinib. Gefitinib is a selective EGFR-tyrosine kinase inhibitor that inhibits EGF-stimulated EGFR autophosphorylation.

Because EGFR is the direct target of a number of anticancer drugs, variable expression of EGFR may directly affect drug response and toxicity. Therefore, polymorphisms in the EGFR gene relevant to gene expression or activity will be important both to further understanding the cell signal transduction and to elucidating drug response/toxicity. Studies of the polymorphisms in the EGFR gene may also be useful for future drug design.

EGFR expression is also associated with diseases other than cancer. For example, an association was reported between an EGFR microsatellite polymorphism and the rate of progression of autosomal dominant polycystic kidney disease (ADPKD) (Magistroni et al., 2003). It has been suggested that mutations that influence the function or expression of EGFR might predispose to inflammatory bowel disease (Martin et al., 2002). Thus, the identification of polymorphisms in the EGFR gene relevant to its expression or activity will be important to further understand the progression of a variety of diseases associated with EGFR dysregulation.

### SUMMARY OF THE INVENTION

The present invention discloses twelve polymorphisms in the EGFR 5′ regulatory region. More particularly, the inventors demonstrated that the −216G>T polymorphism is associated with increased expression from the EGFR promoter region. The identification of polymorphisms associated with EGFR expression enables novel methods and compositions for evaluating the potential efficacy and/or toxicity of an EGFR-targeting therapeutic agent, predicting a patient's clinical prognosis, and evaluating a patient's risk of developing a disease that is associated with EGFR dysregulation.

The present invention discloses polymorphic sites in the EGFR gene locus at nucleotide positions −1435, −1300, −1249, −1227, −761, −650, −544, −486, −216, −191, 169, and 2034. The nucleotide positions −1435, −1300, −1249, −1227, −761, −650, −544, −486, −216, −191, 169, and 2034 of the EGFR gene locus are identified by their position in relation to the translation start site, which is designated +1. There is no nucleotide position designated 0. According to this nomenclature the nucleotide immediately 5′ of +1 is −1, and the nucleotide immediately 3′ of +1 is 2. The translation start site (+1) corresponds to nucleotide 9,385 of the EGFR gene locus (GenBank accession number AF288738, incorporated herein by reference) and nucleotide 505 of SEQ ID NO:1. SEQ ID NO:1 includes nucleotides 8,881 to 9,405 of AF288738.

The specific polymorphism discovered by the inventors are −1435 C>T, −1300 G>A, −1249 G>A, −1227 G>A, −761 C>A, −650 G>A, −544 G>A, −486 C>A, −216 G>T, −191 C>A, 169 G>T, and 2034 G>A. As these polymorphisms are located in the 5′ regulatory region of the EGFR gene, they may be associated with gene regulation.

Thus, in one embodiment, the present invention provides a method for predicting the expression level of EGFR in a cell or cells comprising determining the sequence at one or more of nucleotide positions −1435, −1300, −1249, −1227, −761, −650, −544, −486, −216, −191, 169, or 2034 on one or both EGFR genes in the cell. Consequently, a patient having such cells could be predicted to have that general level of EGFR expression. In a preferred embodiment the method comprises determining the sequence at position −216 in one or both alleles of the EGFR gene in the cell. The presence of a T at position −216 in one or both alleles is indicative of a higher expression level. A “higher expression level” is a level of expression that is greater than the expression level in a cell with a G at position −216 on both alleles of the EGFR gene. The term “determining” is used according to its plain and ordinary meaning; it means to find out or come to a decision about by investigation, reasoning, or calculation.

Polymorphisms in linkage disequilibrium with a polymorphism at nucleotide positions −1435, −1300, −1249, −1227, −761, −650, −544, −486, −216, −191, 169, or 2034 of the EGFR gene locus may also be used with the methods of the present invention. “Linkage disequilibrium” (“LD” as used herein, though also referred to as “LED” in the art) is used according to its plain and ordinary meaning to one skilled in the art. LD refers to a situation where a particular combination of alleles (i.e., a variant form of a given gene) or polymorphisms at two loci appears more frequently than would be expected. “Significant” as used in respect to linkage disequilibrium, as determined by one of skill in the art, is contemplated to be a statistical p or α value that may be 0.25 or 0.1 and may be 0.1, 0.05. 0.001, 0.00001 or less. The relationship between EGFR haplotypes and the expression level of the EGFR protein may be used to correlate the genotype (i.e., the genetic make up of an organism) to a phenotype (i.e., the physical traits displayed by an organism or cell). “Haplotype” is used according to its plain and ordinary meaning to one skilled in the art. It refers to the genotype of two or more alleles or polymorphisms along one of the homologous chromosomes.

The sequences at, or in linkage disequilibrium with, nucleotide positions −1435, −1300, −1249, −1227, −761, −650, −544, −486, −216, −191, 169, and 2034 of the EGFR gene locus may be determined by any method know to those skilled in the art. The sequence may be determined directly or indirectly. The sequence of a nucleotide position of interest may be determined indirectly by, for example, determining the nucleotide sequence at a position known to be in linkage disequilibrium with a specific nucleic acid at the position of interest. Methods for determining the sequence at a specific nucleotide position include, for example, hybridization assays, allele specific amplification assays, sequencing assays, a microsequencing assays, invasive cleavage assays, and restriction enzyme assays. In a specific embodiment, the presence of a −216 G>T polymorphism is determined by digestion with restriction enzyme BseR1. An allele with a T at position −216 can be cut with BseR1, whereas an allele with a G at position −216 cannot be cut.

In other embodiments, the invention provides methods for evaluating the potential efficacy of an EGFR-targeting therapeutic agent for the treatment of a disease associated with the dysregulation of EGFR in a patient comprising determining the sequence at nucleotide position −1435, −1300, −1249, −1227, −761, −650, −544, −486, −216, −191, 169, or 2034 in one or both EGFR genes in the patient.

A disease associated with the dysregulation of EGFR may be any disease in which EGFR is overexpressed, underexpressed, or expressed at inappropriate times compared to the expression in comparable normal cells. Examples of diseases associated with the improper regulation of EGFR expression include cancer, autosomal dominant polycystic kidney disease, and inflammatory disorders such as inflammatory bowel disease.

An EGFR-targeting therapeutic agent may be any agent capable of modulating EGFR activity either directly or indirectly. EGFR-targeting therapeutic agents known in the art are typically directed to inhibiting EGFR phosphorylation or blocking EGF binding. Two EGFR-targeting therapeutic agents have received FDA approval, Iressa (gefitinib) and Erbitux (cetuximab). Another EGFR-targeting therapeutic agent, Tarceva (erlotinib), is in phase III trials. Iressa and Tarceva are small molecules, whereas Erbitux is a monoclonal antibody. Other EGFR-targeting agents modulate EGFR activity by regulating its transcription. For example, EGFR mRNA production can be stimulated directly or indirectly by treating cells with EGF, dexamethasone, thyroid hormone, retinoic acids, interferon a, or wild-type p53.

In certain aspects, the present invention provides methods for evaluating the potential efficacy of an EGFR-targeting therapeutic agent for the treatment of cancer in a patient comprising determining the sequence at nucleotide position −1435, −1300, −1249, −1227, −761, −650, −544, −486, −216, −191, 169, or 2034 in one or both EGFR genes in the patient. In some embodiments the EGFR-targeting therapeutic agent is gefitinib, erlotinib, or cetuximab. In a preferred the sequence at position −216 is determined. In some embodiments, a patient having a T at position −216 on one or both alleles of the EGFR gene is an indicator of decreased efficacy of the EGFR-targeting therapeutic agent as compared to a patient with a G at position −216 on both alleles.

In some embodiments, the methods of the present invention further comprise obtaining a sample. A sample may be any sample containing genomic DNA from which the sequence at nucleotide position −1435, −1300, −1249, −1227, −761, −650, −544, −486, −216, −191, 169, or 2034 in one or both EGFR genes can be determined. The sample may be obtained by, for example, biopsy, venipuncture, aspiration, or swabbing. The sample may be from any tissue or body fluid. In certain embodiments, the sample comprises buccal cells, mononuclear cells, or cancer cells.

In certain aspects, the methods of the present invention further comprise administering the EGFR-targeting therapeutic agent to the patient.

In other embodiments, the present invention provides methods for predicting the clinical prognosis for a patient having a disease associated with the dysregulation of EGFR comprising determining the sequence at, or in linkage disequilibrium with, one or more of nucleotide positions −1435, −1300, −1249, −1227, −761, −650, −544, −486, −216, −191, 169, or 2034 in one or both EGFR genes in the patient. In some embodiments the polymorphism is −216 G>T. The presence of a T at position −216 on an allele is an indicator of an increased expression of EGFR protein. In certain aspects, the increased expression of EGFR protein is predictive of poor prognosis. In some embodiments, the disease associated with the dysregulation of EGFR is cancer. For a patient with cancer, poor prognosis may indicate, for example, increased resistance to chemotherapy, hormonal therapy, or radiotherapy. Poor prognosis may also indicate an increased risk of metastasis or decreased survival time.

In one embodiment, the present invention provides methods for evaluating a patient's risk of toxicity to an EGFR-targeting therapeutic agent comprising determining the presence of a polymorphism at one or more of nucleotide positions −1435, −1300, −1249, −1227, −761, −650, −544, −486, −216, −191, 169, or 2034 in one or both EGFR genes in the patient. In one aspect, the polymorphism is −216 G>T. In one embodiment, the presence of a T at position −216 on one or both alleles is an indicator of decreased toxicity of the EGFR-targeting therapeutic agent.

In other embodiments, the present invention provides methods for evaluating a patient's risk of developing cancer comprising determining the presence of a polymorphism at one or more of nucleotide positions −1435, −1300, −1249, −1227, −761, −650, −544, −486, −216, −191, 169, or 2034 in one or both EGFR genes in the patient. In one embodiment the polymorphism is −216 G>T.

In certain aspects of the present invention, the methods further comprising taking a patient history, wherein the patient is identified as being at risk for developing cancer or in need of an EGFR-targeting therapeutic agent.

The present invention also provides kits. In one embodiment, the present invention provides kits for the detection of a polymorphism at one or more of nucleotide positions −1435, −1300, −1249, −1227, −761, −650, −544, −486, −216, −191, 169, or 2034. In some embodiments, the kit contains a nucleic acid for determining the presence of the polymorphism. The nucleic acid may be a primer or a probe. In some embodiments, the probe is comprised in an oligonucleotide array or microarray. In other embodiments, the kit contains a restriction enzyme for determining the presence of the polymorphism. In certain embodiments, the kit contains both a nucleic acid and a restriction enzyme. A control nucleic acid may be included in the kit.

In some embodiments, the nucleic acids of the kit comprise 7, 8, 9, 10, 11, 12, 13, 14, 15, 16, 17, 18, 19, 20, 21, 22, 23, 24, 25, 30, 40, 50, 60, 70, 80, 90, 100, 200, 300, 400, 500 or more consecutive nucleotides of SEQ ID NO: 2.

In certain aspects, the present invention provides kits for evaluating the potential efficacy of an EGFR-targeting therapeutic agent in a patient comprising a nucleic acid for determining the presence of a polymorphism at nucleotide position −1435, −1300, −1249, −1227, −761, −650, −544, −486, −216, −191, 169, or 2034 in the EGFR gene locus. In other aspects, the present invention provides kits for evaluating the potential efficacy of an EGFR-targeting therapeutic agent in a patient comprising a restriction enzyme for determining the presence of a polymorphism at nucleotide position −1435, −1300, −1249, −1227, −761, −650, −544, −486, −216, −191, 169, or 2034 in the EGFR gene locus.

It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. FIG. 1 is a map of the EGFR locus. The EGFR regulatory region is expanded to show the promoter, enhancers, and exon 1. The location of the 12 single nucleotide polymorphisms discovered in the regulatory region are indicated as arrows.

FIG. 2. FIG. 2 shows the nucleotide sequence of the EGFR promoter region. The nucleotide sequence is from −504 to +21 where +1 designates the first nucleotide of the translation start codon and there is no nucleotide designated 0. The positions of the −216 G>T polymorphism, −191 C>A polymorphism, Sp1 binding site, transcription initiation site, SacI cutting site, and the position of the forward primer are also indicated.

FIG. 3. FIG. 3 shows the vector map constructed for the luciferase activity assays. The 405 bp KpnI-SacI fragment of the EGFR promoter was cloned into the polyclonal site upstream of the luciferase gene. The positions of primers, RVP3 and GLP2, which were used to sequence the cloned fragments, are also indicated.

FIG. 4. FIG. 4 shows the expression activity of the four haplotypes for the EGFR polymorphisms −216 G>T and −191 C>A in transient transfection assays with the luciferase reporter construct. Relative expression of the luciferase gene was normalized by the renilla gene level in the pRL-TK vector.

FIG. 5. FIG. 5 shows an electromobility shift assay testing the binding efficiency of nuclear proteins to the −216G and −216T alleles. The Sp1 consensus probe was used as a control. The probe and competitor sequences used in the EMSA are listed in Table 4. Significantly higher binding efficiency of nuclear protein was observed with the −216T allele (lane 3) compared to the −216G allele (lane 1).

FIG. 6A-B. Transient transfection of pGL3EGFRluc (*1 to *4) in MDA-MB-231, MCF-7, HEK-293 and SL-2 cells (A). For human cell lines, 1.6 μg pGL3EGFRluc was co-transfected with 160 ng pRL-TK vector. For SL-2 cells, 300 ng pGL3EGFRluc was co-transfected with 100 ng pPac-Sp1 vector and relative expression of 200 light units of luciferase activity/μg total protein/ml was set to 1. Significant difference of promoter activity was observed between G-C and T-C haplotype of −216G/T−191C/A (all p values are less than 0.04). Data were shown as mean±SEM. Relative expression of EGFR among MDA-MB-231, MCF-7 and HEK293 cell lines and corresponding genotypes of −216G/T and −191C/A polymorphisms were shown in (B). EGFR mRNA level was normalized to 1000 copies of β-actin gene. Experiments were repeated three times and data were shown as mean±SEM.

### DESCRIPTION OF ILLUSTRATIVE EMBODIMENTS

A. Epidermal Growth Factor Receptor

Human epidermal growth factor receptor (EGFR) is a transmembrane protein. Binding of ligands, such as epidermal growth factor and TGF-α, with its N-terminus on the extracellular surface induces receptor dimerization and activates the tyrosine kinase activity of the intracellular domain. Activation of EGFR leads to a cascade of cellular events that ultimately result in DNA synthesis, and cell proliferation, maturation, survival, and apoptosis.

The expression of EGFR is mainly regulated at the transcription level (Xu et al., 1984). It has been demonstrated that EGFR mRNA production can be stimulated directly or indirectly by treating cells with EGF, dexamethasone, thyroid hormone, retinoic acids, interferon α, or wild-type p53 (Deb et al., 1994; Grandis et al., 1996; Hudson et al., 1989; Subler et al., 1994; Xu et al., 1993.

The EGFR 5′ regulatory region spans about 4 kb covering 2 kb upstream and 2 kb downstream of exon 1. The regulatory elements include a promoter region and two separate enhancer regions. The function of the EGFR promoter and enhancers are well studied and documented (Ishii et al., 1985; Haley et al., 1987; Johnson et al., 1988; Kageyama et al., 1988; Maekawa et al., 1989; each of which in incorporated by reference). Briefly, there is no TATA or CAAT box found in the promoter. Instead, there are multiple transcription initiation sites (Ishii et al., 1985; Haley et al., 1987; Johnson et al., 1988; Kageyama et al., 1988). A number of cis- and trans-regulators have been discovered. These regulators include EGF responsive DNA-binding protein (ERDBP-1), p53, p63, Sp1, Vitamin D-responsive element (VDRE) and estrogen responsive element, which reflects the perplexing regulation of EGFR.

Deoxyribonuclease I footprinting showed that Sp1 can bind to four CCGCCC sequences (−457 to −440, −365 to −286, −214 to −200, and −110 to −84) in the EGFR gene promoter and may, therefore, play a vital role in the gene regulation (Johnson et al., 1998). Studies by Gebhardt and colleagues (1999) demonstrated that a dinucleotide (CA)n repeat polymorphism in the intron 1 of EGFR (near the downstream enhancer) ranging from 14 to 21 repeats, appears to regulate EGFR expression. The longer allele with 21 repeats showed an 80% reduction of gene expression compared to the shorter allele with 16 repeats (Gebhardt et al., 1999; Buerger et al., 2000). Data from studies on the polymorphic CA repeat suggest that this polymorphic site may play a role in cancer susceptibility (Brandt et al., 2004).

Overexpression of EGFR is found in about 30% of human primary tumors. Its activation in these tumors appears to promote tumor growth by increasing cell proliferation, motility, adhesion, invasive capacity, and by blocking apoptosis (Tysnes et al., 1997). EGFR overexpression and dysregulation has been associated with poorer prognosis in patients, and with metastasis, late-stage disease, and resistance to chemotherapy, hormonal therapy, and radiotherapy (Salomon et al., 1995); Akimoto et al., 1999); Wosikowski et al., 2000).

Based on the observation that the overexpression of EGFR is associated with some cancers and that it appears to promote tumor growth, the identification of polymorphisms in the EGFR gene relevant to gene expression may be important for predicting an individual's risk of developing cancer and for predicting a cancer patient's prognosis. In addition, polymorphisms relevant to EGFR expression could also be used to evaluate toxicity, dosage, and potential efficacy of EGFR-targeting agents.

Several EGFR-targeted cancer therapies are currently under development. EGFR-targeting agents are typically directed to inhibiting EGFR phosphorylation or blocking EGF binding. Two EGFR-targeting drugs have been approved, Iressa (gefitinib) and Erbitux (cetuximab), and Tarceva (erlotinib) is in phase III trials. Because EGFR is the direct target of a number of anticancer drugs, variable expression of EGFR may directly affect drug response and toxicity. Therefore, polymorphisms in the EGFR gene relevant to gene expression or activity will be important both to further understanding the cell signal transduction and to elucidating drug response/toxicity. Studies of the polymorphisms in the EGFR gene may also be useful for future drug design.

EGFR expression is also associated with diseases other than cancer. EGFR is a key element in renal tubular proliferation. Recently, an association was reported between an EGFR microsatellite polymorphism and the rate of progression of autosomal dominant polycystic kidney disease (ADPKD) (Magistroni et al., (2003). It was also demonstrated that inhibiting EGFR with a specific tyrosine kinase inhibitor (EKI-785) could slow disease progression in a murine model of ADPKD (Sweeney et al., 1999).

Human EGFR maps to chromosome 7p12, a region that has been linked to inflammatory bowel disease (Satsangi et al., 1996). Furthermore, a marked increase in EGFR immunoreactivity has been observed in animal models of colitis (Reinshagen et al., 1993). It has been suggested that mutations that influence the function or expression of EGFR might predispose to inflammatory bowel disease (Martin et al., 2002).

Given the importance of EGFR in regulating cell proliferation, polymorphisms in the EGFR gene relevant to its expression or activity will be important to further understand the progression of diseases associated with EGFR dysregulation. The present invention has identified 12 polymorphisms in the 5′ regulatory region of the EGFR gene, −1435 C>T, −1300 G>A, −1249 G>A, −1227 G>A, −761 C>A, −650 G>A, −544 G>A, −486 C>A, −216 G>T, −191 C>A, 169 G>T, and 2034 G>A. The polymorphisms are identified in relation to their position from the translation start site, which is designated +1. According to this nomenclature the nucleotide immediately 5′ of +1 is −1, and the nucleotide immediately 3′ of +1 is 2. The translation start site (+1) corresponds to nucleotide 9,385 of the EGFR gene locus (GenBank accession number AF288738) and nucleotide 505 of SEQ ID NO:1. SEQ ID NO:1 includes nucleotides 8,881 to 9,405 of the EGFR gene locus.

One SNP, −1249 G>A is in the upstream enhancer while −216 G>T and −191 C>A are in the promoter region. Interestingly, −216 G>T is located in a Sp1 binding site and the replacement of G by T may alter the Sp1 binding. The −191 C>A is close to a transcription initiation site. Therefore, these SNPs may have a significant impact on the EGFR transcription.

B. Nucleic Acids

Certain embodiments of the present invention concern various nucleic acids, including promoters, amplification primers, oligonucleotide probes and other nucleic acid elements involved in the analysis of genomic DNA. In certain aspects, a nucleic acid comprises a wild-type, a mutant, or a polymorphic nucleic acid.

The term “nucleic acid” is well known in the art. A “nucleic acid” as used herein will generally refer to a molecule (i.e., strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.”

The term “oligonucleotide” refers to a molecule of between about 3 and about 100 nucleobases in length. The term “polynucleotide” refers to at least one molecule of greater than about 100 nucleobases in length. A “gene” refers to coding sequence of a gene product, as well as introns and the promoter of the gene product. In addition to the EGFR gene, other regulatory regions such as the promoter and enhancers for EGFR are contemplated as nucleic acids for use with compositions and methods of the claimed invention.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss”, a double stranded nucleic acid by the prefix “ds”, and a triple stranded nucleic acid by the prefix “ts.”

The term “gene” refers to the segment of DNA involved in producing a polypeptide chain; it includes regions preceding and following the coding region as well as intervening sequences (introns) between individual coding segments (exons). A “promoter” is a region of a nucleic acid sequence at which initiation and rate of transcription are controlled. It may contain elements at which regulatory proteins and molecules may bind, such as RNA polymerase and other transcription factors, to initiate the specific transcription of a nucleic acid sequence. The term “enhancer” refers to a cis-acting regulatory sequence involved in the transcriptional activation of a nucleic acid sequence. An enhancer can function in either orientation and may be upstream or downstream of the promoter.

1. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemical synthesis using phosphotriester, phosphite, or phosphoramidite chemistry and solid phase techniques such as described in European Patent 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotides may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid includes one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

2. Purification of Nucleic Acids

A nucleic acid may be purified on polyacrylamide gels, cesium chloride centrifugation gradients, chromatography columns or by any other means known to one of ordinary skill in the art (see for example, Sambrook et al., 2001, incorporated herein by reference).

In certain aspects, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

3. Nucleic Acid Segments

In certain embodiments, the nucleic acid is a nucleic acid segment. As used herein, the term “nucleic acid segment,” are fragments of a nucleic acid, such as, for a non-limiting example, those that encode only part of a EGFR gene sequence. Thus, a “nucleic acid segment” may comprise any part of a gene sequence, including from about 2 nucleotides to the full length gene including regulatory regions to the polyadenylation signal and any length that includes all the coding region.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:
n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

4. Nucleic Acid Complements

The present invention also encompasses a nucleic acid that is complementary to a nucleic acid. A nucleic acid “complement(s)” or is “complementary” to another nucleic acid when it is capable of base-pairing with another nucleic acid according to the standard Watson-Crick, Hoogsteen, or reverse Hoogsteen binding complementarity rules. As used herein “another nucleic acid” may refer to a separate molecule or a spatially separated sequence of the same molecule. In preferred embodiments, a complement is a hybridization probe or amplification primer for the detection of a nucleic acid polymorphism.

As used herein, the term “complementary” or “complement” also refers to a nucleic acid comprising a sequence of consecutive nucleobases or semiconsecutive nucleobases (e.g.; one or more nucleobase moieties are not present in the molecule) capable of hybridizing to another nucleic acid strand or duplex even if less than all the nucleobases do not base pair with a counterpart nucleobase. However, in some diagnostic or detection embodiments, completely complementary nucleic acids are preferred.

C. Nucleic Acid Detection

Some embodiments of the invention concern identifying polymorphisms in EGFR, correlating genotype or haplotype to phenotype, wherein the phenotype is lowered or altered EGFR activity or expression, and then identifying such polymorphisms in patients who have or will be given EGFR-targeting drugs or compounds. Thus, the present invention involves assays for identifying polymorphisms and other nucleic acid detection methods. Nucleic acids, therefore, have utility as probes or primers for embodiments involving nucleic acid hybridization. They may be used in diagnostic or screening methods of the present invention. Detection of nucleic acids encoding EGFR, as well as nucleic acids involved in the expression or stability of EGFR polypeptides or transcripts, are encompassed by the invention. General methods of nucleic acid detection are provided below, followed by specific examples employed for the identification of polymorphisms, including single nucleotide polymorphisms (SNPs).

1. Hybridization

The use of a probe or primer of between 3, 4, 5, 6, 7, 8, 9, 10, 11, 12, 13, 14, 15, 16, 17, 18, 19, 20, 21, 22, 23, 24, 25, 50, 60, 70, 80, 90, or 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In certain embodiments, the probe or primer comprises 7, 8, 9, 10, 11, 12, 13, 14, 15, 16, 17, 18, 19, 20, 21, 22, 23, 24, 25, 50, 60, 70, 80, 90, or 100 consecutive nucleotides of SEQ ID NO: 1. In some embodiments, the probe or primer comprises 7, 8, 9, 10, 11, 12, 13, 14, 15, 16, 17, 18, 19, 20, 21, 22, 23, 24, 25, 50, 60, 70, 80, 90, or 100 consecutive nucleotides of SEQ ID NO:2.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting a specific polymorphism. It is generally appreciated that conditions can be rendered more stringent by the addition of increasing amounts of formamide. For example, under highly stringent conditions, hybridization to filter-bound DNA may be carried out in 0.5 M NaHPO₄, 7% sodium dodecyl sulfate (SDS), 1 mM EDTA at 65° C., and washing in 0.1×SSC/0.1% SDS at 68° C. (Ausubel et al., 1996).

Conditions may be rendered less stringent by increasing salt concentration and/or decreasing temperature. For example, a medium stringency condition could be provided by about 0.1 to 0.25M NaCl at temperatures of about 37° C. to about 55° C., while a low stringency condition could be provided by about 0.15M to about 0.9M salt, at temperatures ranging from about 20° C. to about 55° C. Under low stringent conditions, such as moderately stringent conditions the washing may be carried out for example in 0.2×SSC/0.1% SDS at 42° C. (Ausubel et al., 1996). Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase, or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples. In other aspects, a particular nuclease cleavage site may be present and detection of a particular nucleotide sequence can be determined by the presence or absence of nucleic acid cleavage.

In general, it is envisioned that the probes or primers described herein will be useful as reagents in solution hybridization, as in PCR™, for detection of expression or genotype of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

2. Amplification of Nucleic Acids

Nucleic acids used as a template for amplification may be isolated from cells, tissues or other samples according to standard methodologies (Sambrook et al., 2001). In certain embodiments, analysis is performed on whole cell or tissue homogenates or biological fluid samples with or without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to the EGFR gene locus (Genbank accession number AF288738) or variants thereof, and fragments thereof are contacted with the template nucleic acid under conditions that permit selective hybridization. SEQ ID NO:1 includes nucleotides 8,881 to 9,405 of the EGFR gene locus with nucleotide 505 of SEQ ID NO:1 corresponding to the translational start site of the EGFR gene, thus the translational start site is located at nucleotide 9,385 of AF288738. Depending upon the desired application, high stringency hybridization conditions may be selected that will only allow hybridization to sequences that are completely complementary to the primers. In other embodiments, hybridization may occur under reduced stringency to allow for amplification of nucleic acids that contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected, analyzed or quantified. In certain applications, the detection may be performed by visual means. In certain applications, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

Another method for amplification is ligase chain reaction (“LCR”), disclosed in European Application No. 320,308, incorporated herein by reference in its entirety. U.S. Pat. No. 4,883,750 describes a method similar to LCR for binding probe pairs to a target sequence. A method based on PCR™ and oligonucleotide ligase assay (OLA) (described in further detail below), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, Great Britain Application 2 202 328, and in PCT Application PCT/US89/01025, each of which is incorporated herein by reference in its entirety. Qbeta Replicase, described in PCT Application PCT/US87/00880, may also be used as an amplification method in the present invention.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) discloses a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1994; Ohara et al., 1989).

3. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al, 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by spin columns and/or chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized, with or without separation. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Other Assays

Other methods for genetic screening may be used within the scope of the present invention, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR™ (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNaseA mismatch cleavage assay that involves annealing single-stranded DNA or RNA test samples to an RNA probe, and subsequent treatment of the nucleic acid duplexes with RNaseA. For the detection of mismatches, the single-stranded products of the RNaseA treatment, electrophoretically separated according to size, are compared to similarly treated control duplexes. Samples containing smaller fragments (cleavage products) not seen in the control duplex are scored as positive.

Other investigators have described the use of RNaseI in mismatch assays. The use of RNaseI for mismatch detection is described in literature from Promega Biotech. Promega markets a kit containing RNaseI that is reported to cleave three out of four known mismatches. Others have described using the MutS protein or other DNA-repair enzymes for detection of single-base mismatches.

Alternative methods for detection of deletion, insertion or substitution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

5. Specific Examples of SNP Screening Methods

Spontaneous mutations that arise during the course of evolution in the genomes of organisms are often not immediately transmitted throughout all of the members of the species, thereby creating polymorphic alleles that co-exist in the species populations. Often polymorphisms are the cause of genetic diseases. Several classes of polymorphisms have been identified. For example, variable nucleotide type polymorphisms (VNTRs), arise from spontaneous tandem duplications of di- or trinucleotide repeated motifs of nucleotides. If such variations alter the lengths of DNA fragments generated by restriction endonuclease cleavage, the variations are referred to as restriction fragment length polymorphisms (RFLPs). RFLPs are widely used in human and animal genetic analyses.

Another class of polymorphisms are generated by the replacement of a single nucleotide. Such single nucleotide polymorphisms (SNPs) rarely result in changes in a restriction endonuclease site. Thus, SNPs are rarely detectable by restriction fragment length analysis. SNPs are the most common genetic variations and occur once every 100 to 300 bases and several SNP mutations have been found that affect a single nucleotide in a protein-encoding gene in a manner sufficient to actually cause a genetic disease. SNP diseases are exemplified by hemophilia, sickle-cell anemia, hereditary hemochromatosis, late-onset alzheimer disease etc.

In context of the present invention, polymorphic mutations that affect the activity and/or levels of the EGFR gene products will be determined by a series of screening methods. One set of screening methods is aimed at identifying SNPs that affect the inducibility, activity and/or level of the EGFR gene products in in vitro or in vivo assays. The other set of screening methods will then be performed to screen an individual for the occurrence of the SNPs identified above. To do this, a sample (such as blood or other bodily fluid or tissue sample) will be taken from a patient for genotype analysis. The presence or absence of SNPs will determine the level of EGFR expression and/or activity. According to methods provided by the invention, these results will be used to adjust and/or alter the dose of the EGFR-targeting therapeutic agent given to an individual in order to reduce drug side effects.

SNPs can be the result of deletions, point mutations and insertions. In general any single base alteration, whatever the cause, can result in a SNP. The greater frequency of SNPs means that they can be more readily identified than the other classes of polymorphisms. The greater uniformity of their distribution permits the identification of SNPs “nearer” to a particular trait of interest. The combined effect of these two attributes makes SNPs extremely valuable. For example, if a particular trait (e.g., overexpression of EGFR) reflects a mutation at a particular locus, then any polymorphism that is linked to the particular locus can be used to predict the probability that an individual will be exhibit that trait. In some cases, the SNP may be the cause of the trait. For example, a SNP in the Sp1 binding site of the EGFR regulatory region may alter Sp1 binding and thus effect transcription of EGFR.

Several methods have been developed to screen polymorphisms and some examples are listed below. The reference of Kwok and Chen (2003) and Kwok (2001) provide overviews of some of these methods; both of these references are specifically incorporated by reference.

SNPs relating to the regulation of EGFR gene expression can be characterized by the use of any of these methods or suitable modification thereof. Such methods include the direct or indirect sequencing of the site, the use of restriction enzymes where the respective alleles of the site create or destroy a restriction site, or the use of allele-specific hybridization probes.

Examples of identifying polymorphisms and applying that information in a way that yields useful information regarding patients can be found, for example, in U.S. Pat. No. 6,472,157; U.S. Patent Application Publications 20020016293, 20030099960, 20040203034; WO 0180896, all of which are hereby incorporated by reference.

a) DNA Sequencing

The most commonly used method of characterizing a polymorphism is direct DNA sequencing of the genetic locus that flanks and includes the polymorphism. Such analysis can be accomplished using either the “dideoxy-mediated chain termination method,” also known as the “Sanger Method” (Sanger et al., 1975) or the “chemical degradation method,” also known as the “Maxam-Gilbert method” (Maxam et al., 1977). Sequencing in combination with genomic sequence-specific amplification technologies, such as the polymerase chain reaction may be utilized to facilitate the recovery of the desired genes (Mullis et al, 1986; European Patent Application 50,424; European Patent Application. 84,796, European Patent Application 258,017, European Patent Application. 237,362; European Patent Application. 201,184; U.S. Pat. Nos. 4,683,202; 4,582,788; and 4,683,194), all of the above incorporated herein by reference.

b) Exonuclease Resistance

Other methods that can be employed to determine the identity of a nucleotide present at a polymorphic site utilize a specialized exonuclease-resistant nucleotide derivative (U.S. Pat. No. 4,656,127). A primer complementary to an allelic sequence immediately 3′-to the polymorphic site is hybridized to the DNA under investigation. If the polymorphic site on the DNA contains a nucleotide that is complementary to the particular exonucleotide-resistant nucleotide derivative present, then that derivative will be incorporated by a polymerase onto the end of the hybridized primer. Such incorporation makes the primer resistant to exonuclease cleavage and thereby permits its detection. As the identity of the exonucleotide-resistant derivative is known one can determine the specific nucleotide present in the polymorphic site of the DNA.

c) Microsequencing Methods

Several other primer-guided nucleotide incorporation procedures for assaying polymorphic sites in DNA have been described (Komher et al., 1989; Sokolov 1990; Syvanen 1990; Kuppuswamy et al., 1991; Prezant et al., 1992; Ugozzoll et al., 1992; Nyren et al., 1993). These methods rely on the incorporation of labeled deoxynucleotides to discriminate between bases at a polymorphic site. As the signal is proportional to the number of deoxynucleotides incorporated, polymorphisms that occur in runs of the same nucleotide result in a signal that is proportional to the length of the run (Syvanen et al., 1990).

d) Extension in Solution

French Patent 2,650,840 and PCT Application WO91/02087 discuss a solution-based method for determining the identity of the nucleotide of a polymorphic site. According to these methods, a primer complementary to allelic sequences immediately 3′-to a polymorphic site is used. The identity of the nucleotide of that site is determined using labeled dideoxynucleotide derivatives which are incorporated at the end of the primer if complementary to the nucleotide of the polymorphic site.

e) Genetic Bit Analysis or Solid-Phase Extension

PCT Application WO92/15712 describes a method that uses mixtures of labeled terminators and a primer that is complementary to the sequence 3′ to a polymorphic site. The labeled terminator that is incorporated is complementary to the nucleotide present in the polymorphic site of the target molecule being evaluated and is thus identified. Here the primer or the target molecule is immobilized to a solid phase.

f) Oligonucleotide Ligation Assay (OLA)

This is another solid phase method that uses different methodology (Landegren et al., 1988). Two oligonucleotides, capable of hybridizing to abutting sequences of a single strand of a target DNA are used. One of these oligonucleotides is biotinylated while the other is detectably labeled. If the precise complementary sequence is found in a target molecule, the oligonucleotides will hybridize such that their termini abut, and create a ligation substrate. Ligation permits the recovery of the labeled oligonucleotide by using avidin. Other nucleic acid detection assays, based on this method, combined with PCR have also been described (Nickerson et al., 1990). Here PCR is used to achieve the exponential amplification of target DNA, which is then detected using the OLA.

g) Ligase/Polymerase-Mediated Genetic Bit Analysis

U.S. Pat. No. 5,952,174 describes a method that also involves two primers capable of hybridizing to abutting sequences of a target molecule. The hybridized product is formed on a solid support to which the target is immobilized. Here the hybridization occurs such that the primers are separated from one another by a space of a single nucleotide. Incubating this hybridized product in the presence of a polymerase, a ligase, and a nucleoside triphosphate mixture containing at least one deoxynucleoside triphosphate allows the ligation of any pair of abutting hybridized oligonucleotides. Addition of a ligase results in two events required to generate a signal, extension and ligation. This provides a higher specificity and lower “noise” than methods using either extension or ligation alone and unlike the polymerase-based assays, this method enhances the specificity of the polymerase step by combining it with a second hybridization and a ligation step for a signal to be attached to the solid phase.

h) Invasive Cleavage Reactions

Invasive cleavage reactions can be used to evaluate cellular DNA for a particular polymorphism. A technology called INVADER® employs such reactions (e.g., de Arruda et al., 2002; Stevens et al., 2003, which are incorporated by reference). Generally, there are three nucleic acid molecules: 1) an oligonucleotide upstream of the target site (“upstream oligo”), 2) a probe oligonucleotide covering the target site (“probe”), and 3) a single-stranded DNA with the the target site (“target”). The upstream oligo and probe do not overlap but they contain contiguous sequences. The probe contains a donor fluorophore, such as fluoroscein, and an acceptor dye, such as Dabcyl. The nucleotide at the 3′ terminal end of the upstream oligo overlaps (“invades”) the first base pair of a probe-target duplex. Then the probe is cleaved by a structure-specific 5′ nuclease causing separation of the fluorophore/quencher pair, which increases the amount of fluorescence that can be detected. See Lu et al., 2004.

In some cases, the assay is conducted on a solid-surface or in an array format.

h) Other Methods To Detect SNPs

Several other specific methods for SNP detection and identification are presented below and may be used as such or with suitable modifications in conjunction with identifying polymorphisms of the EGFR gene in the present invention. Several other methods are also described on the SNP web site of the NCBI at the website www.ncbi.nlm.nih.gov/SNP, incorporated herein by reference.

In a particular embodiment, extended haplotypes may be determined at any given locus in a population, which allows one to identify exactly which SNPs will be redundant and which will be essential in association studies. The latter is referred to as ‘haplotype tag SNPs (htSNPs)’, markers that capture the haplotypes of a gene or a region of linkage disequilibrium. See Johnson et al. (2001) and Ke and Cardon (2003), each of which is incorporated herein by reference, for exemplary methods.

The VDA-assay utilizes PCR amplification of genomic segments by long PCR methods using TaKaRa LA Taq reagents and other standard reaction conditions. The long amplification can amplify DNA sizes of about 2,000-12,000 bp. Hybridization of products to variant detector array (VDA) can be performed by an Affymetrix High Throughput Screening Center and analyzed with computerized software.

A method called Chip Assay uses PCR amplification of genomic segments by standard or long PCR protocols. Hybridization products are analyzed by VDA, Halushka et al, 1999, incorporated herein by reference. SNPs are generally classified as “Certain” or “Likely” based on computer analysis of hybridization patterns. By comparison to alternative detection methods such as nucleotide sequencing, “Certain” SNPs have been confirmed 100% of the time; and “Likely” SNPs have been confirmed 73% of the time by this method.

Other methods simply involve PCR amplification following digestion with the relevant restriction enzyme. Yet others involve sequencing of purified PCR products from known genomic regions.

In yet another method, individual exons or overlapping fragments of large exons are PCR-amplified. Primers are designed from published or database sequences and PCR-amplification of genomic DNA is performed using the following conditions: 200 ng DNA template, 0.5 μM each primer, 80 μM each of dCTP, dATP, dTTP and dGTP, 5% formamide, 1.5 mM MgCl₂, 0.5 U of Taq polymerase and 0.1 volume of the Taq buffer. Thermal cycling is performed and resulting PCR-products are analyzed by PCR-single strand conformation polymorphism (PCR-SSCP) analysis, under a variety of conditions, e.g., 5 or 10% polyacrylamide gel with 15% urea, with or without 5% glycerol. Electrophoresis is performed overnight. PCR-products that show mobility shifts are reamplified and sequenced to identify nucleotide variation.

In a method called CGAP-GAI (DEMIGLACE), sequence and alignment data (from a PHRAP.ace file), quality scores for the sequence base calls (from PHRED quality files), distance information (from PHYLIP dnadist and neighbour programs) and base-calling data (from PHRED ‘-d’ switch) are loaded into memory. Sequences are aligned and examined for each vertical chunk (‘slice’) of the resulting assembly for disagreement. Any such slice is considered a candidate SNP (DEMIGLACE). A number of filters are used by DEMIGLACE to eliminate slices that are not likely to represent true polymorphisms. These include filters that: (i) exclude sequences in any given slice from SNP consideration where neighboring sequence quality scores drop 40% or more; (ii) exclude calls in which peak amplitude is below the fifteenth percentile of all base calls for that nucleotide type; (iii) disqualify regions of a sequence having a high number of disagreements with the consensus from participating in SNP calculations; (iv) remove from consideration any base call with an alternative call in which the peak takes up 25% or more of the area of the called peak; (v) exclude variations that occur in only one read direction. PHRED quality scores were converted into probability-of-error values for each nucleotide in the slice. Standard Bayesian methods are used to calculate the posterior probability that there is evidence of nucleotide heterogeneity at a given location.

In a method called CU-RDF (RESEQ), PCR amplification is performed from DNA isolated from blood using specific primers for each SNP, and after typical cleanup protocols to remove unused primers and free nucleotides, direct sequencing using the same or nested primers.

In a method called DEBNICK (METHOD-B), a comparative analysis of clustered EST sequences is performed and confirmed by fluorescent-based DNA sequencing. In a related method, called DEBNICK (METHOD-C), comparative analysis of clustered EST sequences with phred quality >20 at the site of the mismatch, average phred quality >=20 over 5 bases 5′-FLANK and 3′ to the SNP, no mismatches in 5 bases 5′ and 3′ to the SNP, at least two occurrences of each allele is performed and confirmed by examining traces.

In a method identified as ERO (RESEQ), new primers sets were designed for electronically published STSs and used to amplify DNA from 10 different mouse strains. The amplification product from each strain is then gel purified and sequenced using a standard dideoxy, cycle sequencing technique with 33P-labeled terminators. All the ddATP terminated reactions are then loaded in adjacent lanes of a sequencing gel followed by all of the ddGTP reactions and so on. SNPs are identified by visually scanning the radiographs.

In another method identified as ERO (RESEQ-HT), new primers sets were designed for electronically published murine DNA sequences and used to amplify DNA from 10 different mouse strains. The amplification product from each strain is prepared for sequencing by treating with Exonuclease I and Shrimp Alkaline Phosphatase. Sequencing is performed using ABI Prism Big Dye Terminator Ready Reaction Kit (Perkin-Elmer) and sequence samples are run on the 3700 DNA Analyzer (96 Capillary Sequencer).

FGU-CBT (SCA2-SNP) identifies a method where the region containing the SNP is PCR amplified using the primers SCA2-FP3 and SCA2-RP3. Approximately 100 ng of genomic DNA is amplified in a 50 ml reaction volume containing a final concentration of 5 mM Tris, 25 mM KCl0.75 mM MgCl₂, 0.05% gelatin, 20 pmol of each primer and 0.5 U of Taq DNA polymerase. Samples are denatured, annealed and extended and the PCR product is purified from a band cut out of the agarose gel using, for example, the QIAquick gel extraction kit (Qiagen) and is sequenced using dye terminator chemistry on an ABI Prism 377 automated DNA sequencer with the PCR primers.

In a method identified as JBLACK (SEQ/RESTRICT), two independent PCR reactions are performed with genomic DNA. Products from the first reaction are analyzed by sequencing, indicating a unique FspI restriction site. The mutation is confirmed in the product of the second PCR reaction by digesting with Fsp I.

In a method described as KWOK(1), SNPs are identified by comparing high quality genomic sequence data from four randomly chosen individuals by direct DNA sequencing of PCR products with dye-terminator chemistry (see Kwok et al., 1996). In a related method identified as KWOK (2) SNPs are identified by comparing high quality genomic sequence data from overlapping large-insert clones such as bacterial artificial chromosomes (BACs) or P1-based artificial chromosomes (PACs). An STS containing this SNP is then developed and the existence of the SNP in various populations is confirmed by pooled DNA sequencing (see Taillon-Miller et al., 1998). In another similar method called KWOK(3), SNPs are identified by comparing high quality genomic sequence data from overlapping large-insert clones BACs or PACs. The SNPs found by this approach represent DNA sequence variations between the two donor chromosomes but the allele frequencies in the general population have not yet been determined. In method KWOK(5), SNPs are identified by comparing high quality genomic sequence data from a homozygous DNA sample and one or more pooled DNA samples by direct DNA sequencing of PCR products with dye-terminator chemistry. The STSs used are developed from sequence data found in publicly available databases. Specifically, these STSs are amplified by PCR against a complete hydatidiform mole (CHM) that has been shown to be homozygous at all loci and a pool of DNA samples from 80 CEPH parents (see Kwok et al., 1994).

In another such method, KWOK (OverlapSnpDetectionWithPolyBayes), SNPs are discovered by automated computer analysis of overlapping regions of large-insert human genomic clone sequences. For data acquisition, clone sequences are obtained directly from large-scale sequencing centers. This is necessary because base quality sequences are not present/available through GenBank. Raw data processing involves analysis of clone sequences and accompanying base quality information for consistency. Finished (‘base perfect’, error rate lower than 1 in 10,000 bp) sequences with no associated base quality sequences are assigned a uniform base quality value of 40 (1 in 10,000 bp error rate). Draft sequences without base quality values are rejected. Processed sequences are entered into a local database. A version of each sequence with known human repeats masked is also stored. Repeat masking is performed with the program “MASKERAID.” Overlap detection: Putative overlaps are detected with the program “WUBLAST.” Several filtering steps follow in order to eliminate false overlap detection results, i.e. similarities between a pair of clone sequences that arise due to sequence duplication as opposed to true overlap. Total length of overlap, overall percent similarity, number of sequence differences between nucleotides with high base quality value “high-quality mismatches.” Results are also compared to results of restriction fragment mapping of genomic clones at Washington University Genome Sequencing Center, finisher's reports on overlaps, and results of the sequence contig building effort at the NCBI. SNP detection: Overlapping pairs of clone sequence are analyzed for candidate SNP sites with the ‘POLYBAYES’ SNP detection software. Sequence differences between the pair of sequences are scored for the probability of representing true sequence variation as opposed to sequencing error. This process requires the presence of base quality values for both sequences. High-scoring candidates are extracted. The search is restricted to substitution-type single base pair variations. Confidence score of candidate SNP is computed by the POLYBAYES software.

In a method identified by KWOK (TaqMan assay), the TaqMan assay is used to determine genotypes for 90 random individuals. In a method identified by KYUGEN(Q1), DNA samples of indicated populations are pooled and analyzed by PLACE-SSCP. Peak heights of each allele in the pooled analysis are corrected by those in a heterozygote, and are subsequently used for calculation of allele frequencies. Allele frequencies higher than 10% are reliably quantified by this method. Allele frequency=0 (zero) means that the allele was found among individuals, but the corresponding peak is not seen in the examination of pool. Allele frequency=0−0.1 indicates that minor alleles are detected in the pool but the peaks are too low to reliably quantify.

In yet another method identified as KYUGEN (Method1), PCR products are post-labeled with fluorescent dyes and analyzed by an automated capillary electrophoresis system under SSCP conditions (PLACE-SSCP). Four or more individual DNAs are analyzed with or without two pooled DNA (Japanese pool and CEPH parents pool) in a series of experiments. Alleles are identified by visual inspection. Individual DNAs with different genotypes are sequenced and SNPs identified. Allele frequencies are estimated from peak heights in the pooled samples after correction of signal bias using peak heights in heterozygotes. The PCR primers are tagged to have 5′-ATT or 5′-GTT at their ends for post-labeling of both strands. Samples of DNA (10 ng/ul) are amplified in reaction mixtures containing the buffer (10 mM Tris-HCl, pH 8.3 or 9.3, 50 mM KCl, 2.0 mM MgCl₂), 0.25 μM of each primer, 200 μM of each dNTP, and 0.025 units/μl of Taq DNA polymerase premixed with anti-Taq antibody. The two strands of PCR products are differentially labeled with nucleotides modified with R110 and R6G by an exchange reaction of Klenow fragment of DNA polymerase I. The reaction is stopped by adding EDTA, and unincorporated nucleotides are dephosphorylated by adding calf intestinal alkaline phosphatase. For the SSCP: an aliquot of fluorescently labeled PCR products and TAMRA-labeled internal markers are added to deionized formamide, and denatured. Electrophoresis is performed in a capillary using an ABI Prism 310 Genetic Analyzer. Genescan softwares (P-E Biosystems) are used for data collection and data processing. DNA of individuals including those who showed different genotypes on SSCP are subjected for direct sequencing using big-dye terminator chemistry, on ABI Prism 310 sequencers. Multiple sequence trace files obtained from ABI Prism 310 are processed and aligned by Phred/Phrap and viewed using Consed viewer. SNPs are identified by PolyPhred software and visual inspection.

In yet another method identified as KYUGEN (Method2), individuals with different genotypes are searched by denaturing HPLC (DHPLC) or PLACE-SSCP (Inazuka et al., 1997) and their sequences are determined to identify SNPs. PCR is performed with primers tagged with 5′-ATT or 5′-GTT at their ends for post-labeling of both strands. DHPLC analysis is carried out using the WAVE DNA fragment analysis system (Transgenomic). PCR products are injected into DNASep column, and separated under the conditions determined using WAVEMaker program (Transgenomic). The two strands of PCR products that are differentially labeled with nucleotides modified with R110 and R6G by an exchange reaction of Klenow fragment of DNA polymerase I. The reaction is stopped by adding EDTA, and unincorporated nucleotides are dephosphorylated by adding calf intestinal alkaline phosphatase. SSCP followed by electrophoresis is performed in a capillary using an ABI Prism 310 Genetic Analyzer. Genescan softwares (P-E Biosystems). DNA of individuals including those who showed different genotypes on DHPLC or SSCP are subjected for direct sequencing using big-dye terminator chemistry, on ABI Prism 310 sequencer. Multiple sequence trace files obtained from ABI Prism 310 are processed and aligned by Phred/Phrap and viewed using Consed viewer. SNPs are identified by PolyPhred software and visual inspection. Trace chromatogram data of EST sequences in Unigene are processed with PHRED. To identify likely SNPs, single base mismatches are reported from multiple sequence alignments produced by the programs PHRAP, BRO and POA for each Unigene cluster. BRO corrected possible misreported EST orientations, while POA identified and analyzed non-linear alignment structures indicative of gene mixing/chimeras that might produce spurious SNPs. Bayesian inference is used to weigh evidence for true polymorphism versus sequencing error, misalignment or ambiguity, misclustering or chimeric EST sequences, assessing data such as raw chromatogram height, sharpness, overlap and spacing; sequencing error rates; context-sensitivity; cDNA library origin, etc.

In method identified as MARSHFIELD (Method-B), overlapping human DNA sequences which contained putative insertion/deletion polymorphisms are identified through searches of public databases. PCR primers which flanked each polymorphic site are selected from the consensus sequences. Primers are used to amplify individual or pooled human genomic DNA. Resulting PCR products are resolved on a denaturing polyacrylamide gel and a Phosphorimager is used to estimate allele frequencies from DNA pools.

6. Linkage Disequilibrium

Polymorphisms in linkage disequilibrium with the polymorphism at −1435, −1300, −1249, −1227, −761, −650, −544, −486, −216, −191, 169, or 2034 of the EGFR gene locus may also be used with the methods of the present invention. “Linkage disequilibrium” (“LD” as used herein, though also referred to as “LED” in the art) refers to a situation where a particular combination of alleles (i.e., a variant form of a given gene) or polymorphisms at two loci appears more frequently than would be expected by chance. “Significant” as used in respect to linkage disequilibrium, as determined by one of skill in the art, is contemplated to be a statistical p or α value that may be 0.25 or 0.1 and may be 0.1, 0.05. 0.001, 0.00001 or less. The relationship between EGFR haplotypes and the expression level of the EGFR protein may be used to correlate the genotype (i.e., the genetic make up of an organism) to a phenotype (i.e., the physical traits displayed by an organism or cell). “Haplotype” is used according to its plain and ordinary meaning to one skilled in the art. It refers to a collective genotype of two or more alleles or polymorphisms along one of the homologous chromosomes.

D. Kits

Any of the compositions described herein may be comprised in a kit. In a non-limiting example, reagents for determining the genotype of one or both EGFR genes are included in a kit. The kit may further include individual nucleic acids that can amplify and/or detect particular nucleic acid sequences the EGFR gene. In specific embodiments, it includes one or more primers and/or probes. Nucleic acid molecules may have a label, dye, or other signalling molecule attached to it, such as a fluorophore. It may also include one or more buffers, such as a DNA isolation buffers, an amplification buffer or a hybridization buffer. The kit may also contain compounds and reagents to prepare DNA templates and isolate DNA from a sample. The kit may also include various labeling reagents and compounds.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

A kit will also include instructions for employing the kit components as well the use of any other reagent not included in the kit. Instructions may include variations that can be implemented.

It is contemplated that such reagents are embodiments of kits of the invention. Such kits, however, are not limited to the particular items identified above and may include any reagent used directly or indirectly in the detection of polymorphisms in the EGFR gene or the expression level of the EGFR gene.

### E. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Discovery of Single Nucleotide Polymorphisms (SNPs) in EGFR Regulatory Region

DNA samples from Coriell Cell Repository were used for resequencing. The samples include 22 Caucasians, 23 African-Americans and 23 Asians. For SNP discovery, PCR was used to amplify the approximately 4.5 kb fragment containing the upstream and downstream enhancer, promoter, exon 1 and part of intron 1 using the primers in Table 1. Purified PCR products were directly sequenced from both ends. ABI-3700 capillary sequencer and a phred/phrap/polyphred/consed pipeline (World Wide Web at phrap.org/) were used to identify the polymorphisms.

TABLE 1

SEQ ID NO:

| | | |
|---|---|---|
| 3 | EGFR1L-F | GTTCCACTGTTGTGCTTCCC |
| 4 | EGFR1L-R | AAGAAAGTTGGGAGCGGTTC |
| 5 | EGFR1L-AF | GGGTGGACTTGCCAAAGGA |
| 6 | EGFR1L-AR | CTTAGAGCCAGCGTCGGATA |
| 7 | EGFR1L-1F | GCATGACTTCAACGCACAGT |
| 8 | EGFR1L-1R | GAGGCTAAGTGTCCCACTGC |
| 9 | EGFR1L-2F | TCGGACTTTAGAGCACCACC |
| 10 | EGFR1L-2R | GAGGAGGAGAATGCGAGGAG |
| 11 | EGFR1L-3F | AAATTAACTCCTCAGGGCACC |
| 12 | EGE11L-3R | CGCCCTTACCTTTCTTTTCC |
| 13 | EGFR1L-4F | CCCTGACTCCGTCCAGTATT |
| 14 | EGFR2L-F | CGTCCTTTCCTGTTTCCTTG |
| 15 | EGFR2L-R | ACCAGCTGTGGGAAAGTCAC |
| 16 | EGFR2L-1R | AGACGAGTTCTCCCAGCTCC |
| 17 | EGFR2L-2F | GCGCAGGTCTCAAACTGAAG |
| 18 | EGFR2L-2R | GGAGAAGTTTGCTGTGAGCC |
| 19 | EGFR2L-3F | CCCTCGTCTTGCCTATCCA |
| 20 | EGFR2L-3R | AGTGATCCCCAAATCTGGCT |
| 21 | EGFR2L-4F | GGCATAGAACAGTGGTTCCC |
| 22 | EGFR2L-4R | GAACACCAATGGAGGGAGAA |
| 23 | EGFR2L-5F | TGAAGGAACTGGTGGAAAGG |
| 24 | EGFR2L-5R | CATGTCCCAGAACCAAACAA |

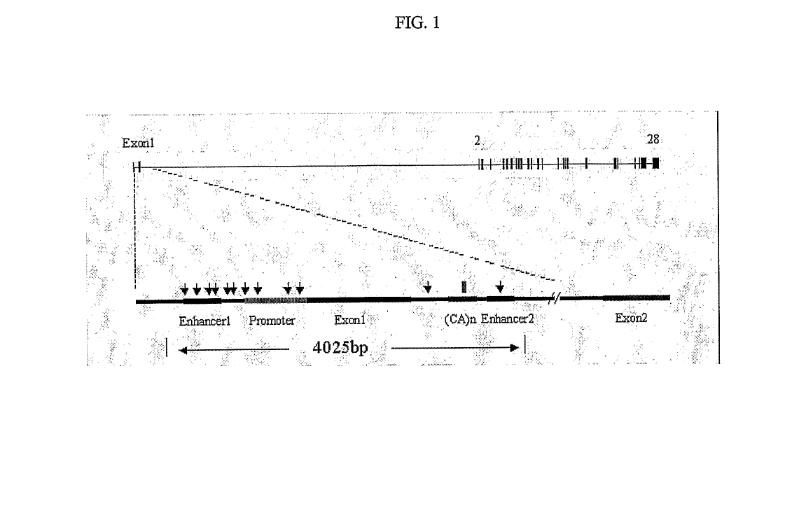

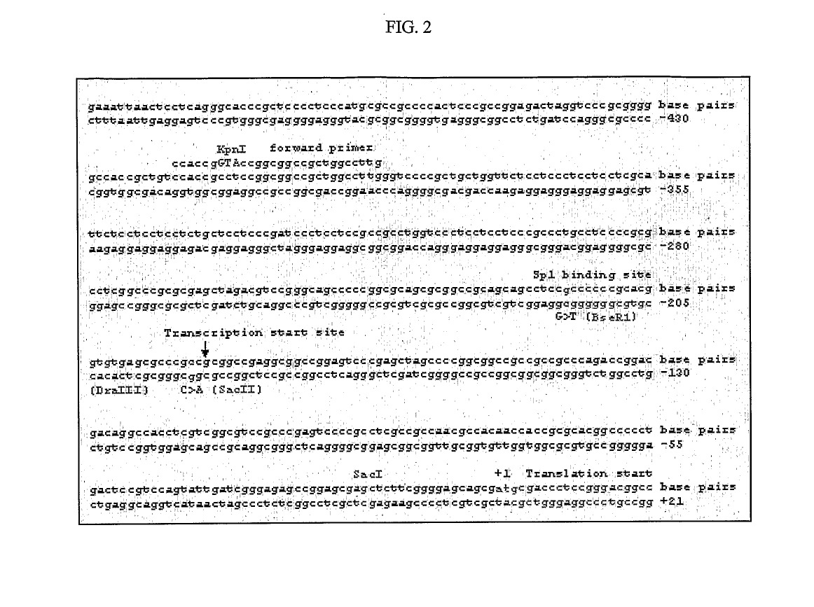

By resequencing 4 kb of the EGFR 5′ regulatory region, including the promoter and enhancers, twelve single nucleotide polymorphisms were identified from 68 DNA samples consisting of 22 Caucasians, 23 African-Americans and 23 Asians (FIG. 1 and Table 2). Five SNPs showed relatively higher frequency (rare allele frequency>10%) at least in one population compared to the other seven rare ones. Nine SNPs were observed in the promoter or enhancer regions and three of these were frequent. One SNP, −1249 G>A (10% in African-Americans) is in the upstream enhancer while −216 G>T (29% in African-Americans and 34% in Caucasians) and −191 C>A are in the promoter region (18% in Caucasian) (FIG. 1 and Table 2). Interestingly, −216 G>T is located in a Sp1 binding site (−216) and the replacement of G by T may alter the Sp1 binding. Meanwhile, the −191 C>A is close to a transcription initiation site (FIG. 2)(Ishii et al., 1985; Haley et al., 1987; Johnson et al., 1988; Kageyama et al., 1988). Therefore, these SNPs may have a significant impact on the EGFR transcription.

TABLE 2
Number and frequency of rare alleles of SNPs discovered from EGFR regulatory region. African-American (AA), Caucasian (CA), Asian (AA).

| | −1435 | | −1300 | | −1249 | | −1227 | | −761 | | −650 | | −544 | | −486 | | −216 | | −191 | | 169 | | 2034 | |
|---|---|---|---|---|---|---|---|---|---|---|---|---|---|---|---|---|---|---|---|---|---|---|---|---|
| | C | T | G | A | G | A | G | A | C | A | G | A | G | A | C | A | G | T | C | A | G | T | G | A |
| AA | 46 | 2 | 46 | 2 | 43 | 5 | 48 | 0 | 41 | 7 | 46 | 2 | 48 | 0 | 45 | 3 | 34 | 14 | 48 | 0 | 42 | 6 | 46 | 2 |
| CA | 44 | 0 | 44 | 0 | 44 | 0 | 43 | 1 | 38 | 6 | 44 | 0 | 44 | 0 | 44 | 0 | 30 | 14 | 38 | 6 | 39 | 5 | 43 | 1 |
| AS | 42 | 0 | 42 | 0 | 42 | 0 | 42 | 0 | 42 | 0 | 42 | 0 | 41 | 1 | 42 | 0 | 39 | 3 | 42 | 0 | 42 | 0 | 38 | 4 |
| AA | | 0.04 | | 0.04 | | 0.1 | | 0 | | 0.15 | | 0.04 | | 0 | | 0.06 | | 0.29 | | 0 | | 0.13 | | 0.04 |
| CA | | 0 | | 0 | | 0 | | 0.02 | | 0.14 | | 0 | | 0 | | 0 | | 0.32 | | 0.14 | | 0.11 | | 0.02 |
| AS | | 0 | | 0 | | 0 | | 0 | | 0 | | 0 | | 0.02 | | 0 | | 0.07 | | 0 | | 0 | | 0.09 |

### Example 2

### Functional Characterization of Two Promoter SNPs (−216G>T and −191 C>A).

Potential function of two SNPs (−216 G>T and −191 C>A) in the EGFR promoter region were characterized by in vitro transient transfection assay and electrophoretic mobility shift assay (EMSA).

Haplotypes.

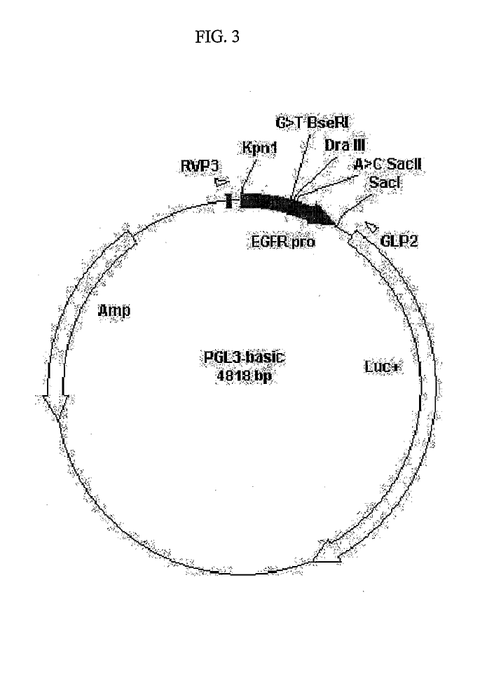

The SNP −216 G>T was found to be frequent in African-Americans (29%) and Caucasians (34%) but relatively rare in Asians (9%), while −191 C>A was only found in Caucasians (18%) when the inventors sequenced the 68 samples from different ethnic groups (Table 3). Linkage disequilibrium and haplotype analysis showed that −216 G>T and −191 C>A are not in strong LD (D′=0.5562, p>0.05,) and three haplotypes were observed in the samples: G-C, G-A and T-C, see Table 3 below. DNA fragments containing these three haplotypes were amplified and cloned while the T-A haplotype was constructed by ligating the T fragment and A fragment from the Dra III digested G-A and T-C haplotypes (FIG. 3).

TABLE 3
The haplotype frequency of −216G > T and −191C > A
in Caucasian, African-American and Asian populations.

| Haplotype | Caucasian | African-American | Asian |
|---|---|---|---|
| G-C | 0.48 | 0.71 | 0.92 |
| G-A | 0.18 | 0.00 | 0.00 |
| T-C | 0.34 | 0.29 | 0.08 |
| T-A | 0.00 | 0.00 | 0.00 |

Vectors and Detecting System.

PGL3-luc+ basic reporter vector (Promega) carrying each of the four target DNA fragments and pRL-TK reporter vector (Promega) containing the renilla gene driven by the herpes simplex virus thymidine kinase (HSV-TK) promoter were co-transfected into MDA-MB-231 cells to compare the relative expression of the luciferase gene. A Dual-Luciferase reporter assay system (Promega) was used to detect the expression level of luciferase. PGL3-luc+ basic vector and PGL3-luc+ SV40-promoter vector were used as negative and positive controls, respectively.

Deletion mapping studies have shown that a 384 bp fragment upstream of exon 1 containing these two SNPs has the essential promoter function (FIG. 2) (Johnson et al., 1988). This fragment was therefore amplified from the individuals with specific haplotypes by PCR using Proofstart DNA polymerase (Qiagen), which is modified for high-fidelity DNA amplification. Primers were designed to amplify the 515 bp amplicon indicated in FIG. 2. The primer sequence was forward primer: 5′-CCACCGGTACCGGCGGCCGCTGGCCTTG-3′ (SEQ ID NO: 25) and reverse primer: 5′-CGGCGAGACACGCCCTTACCTTT-3′ (SEQ ID NO: 26). This 515 bp amplicon contains a SacI cutting site at 3′ end (FIG. 2). To facilitate the subcloning, the forward primer was designed to contain a KpnI site. The fragment was digested by KpnI and SacI and a 405 bp product was then cloned into the KpnI/SacI site of pGL3-luc+ basic vector. To confirm the inserted DNA fragments, all plasmids were sequenced to exclude PCR errors, check the orientation of the fragment, and assure the haplotypes before transfection.

Transient transfection.

The MDA-MB-231 cell line was maintained in RPMI1640 media (Invitrogen) with 10% FBS and 2 mM L-Glutamine. Transient transfection was performed by Transfectamine2000 (Invitrogen) according to the manufacture's instructions. All transfections were performed in triplicate, and repeated three times. Cells were co-transfected with pRL-TK vector to normalize the transfection efficiency. After transfection, cells were cultured for 24 hours, washed, lysed, and analyzed using the Dual Luciferase kit (Promega) according to the manufacturer's instructions.

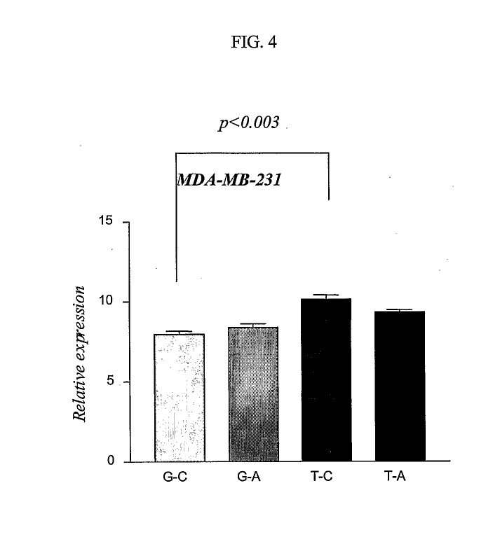

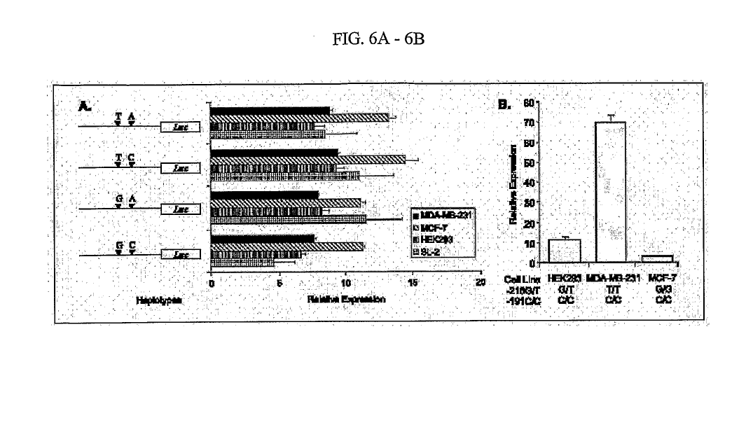

The in vitro transcriptional efficiency of luciferase driven by the four haplotypes were compared. Significantly higher luciferase activity in the T-C haplotype vector was observed than in the G-C haplotype vector (FIG. 4, p<0.01). The T-C and G-C haplotypes are the most frequent haplotypes in Caucasian, African-American, and Asian populations (Table 3). In addition, the −216 G>T polymorphism contributed more to luciferase activity than the −191 C>A polymorphism (FIG. 4; FIG. 6A p<0.04 for all comparisons). This effect was independent of the EGFR expression level of the cells (FIG. 6B). On average, the substitution of the G allele by the T allele demonstrated about a 30% increase in luciferase gene expression.

To further confirm the potential cooperative effect of the DNA alteration and Sp1 on promoter activity, transient transfection was also performed in the Drosophila melanogaster Schneider cell line 2 (SL-2) in which Sp1 is deficient (Courey et al., 1988). As a result, co-transfection of pGL3EGFRluc with Sp1 expression vector resulted in about 100-fold induction of promoter activity compared to transfection of pGL3EGFRluc alone. Co-transfection of pPac-Sp1 and each of four pGL3EGFRluc constructs demonstrated a significantly lower promoter activity driven by G-C haplotype compared to the T-C haplotype (p<0.03. FIG. 6A).

Electrophoretic Mobility Shift Assay (EMSA).

EMSA was used to evaluate nuclear protein binding at the −216G>T polymorphic site. Nuclear proteins were extracted from MDA-MB-231 cells using the NE-PER Nuclear and Cytoplasmic Extraction Reagents according to the manufacture's protocol (Pierce, Rockford, USA). The probes and competitors corresponding to the G allele, T allele, and Sp1 binding consensus sequence are listed in Table 4.

TABLE 4
Probes and competitors used for EMSA.

| | |
|---|---|
| Name | Sequence |

| | |
|---|---|
| G allele probe | |
| GPF (SEQ ID NO:27): | 5′-biotin-GCAGCCTCCGCCCCCCGCACGGTGT-3′ |
| GPR (SEQ ID NO:28): | 5′-biotin-ACACCGTGCGGGGGGCGGAGGCTGC-3′ |
| G allele Competitor | |
| GCF (SEQ ID NO:29): | 5′-GCAGCCTCCGCCCCCCGCACGGTGT-3′ |
| GCR (SEQ ID NO:30): | 5′-ACACCGTGCGGGGGGCGGAGGCTGC-3′ |
| T allele probe | |
| TPF (SEQ ID NO:31): | 5′-biotin-GCAGCCTCCTCCCCCCGCACGGTGT-3′ |
| TPR (SEQ ID NO:32): | 5′-biotin-ACACCGTGCGGGGGGAGGAGGCTGC-3′ |
| T allele Competitor | |
| TCF (SEQ ID NO:33): | 5′-GCAGCCTCCTCCCCCCGCACGGTGT-3′ |
| TCR (SEQ ID NO:34): | 5′-ACACCGTGCGGGGGGAGGAGGCTGC-3′ |
| Sp1 control probe | |
| Sp1PF (SEQ ID NO:35): | 5′-biotin-ATTCGATCGGGGCGGGGCGAGC-3′ |
| Sp1PR (SEQ ID NO:36): | 5′-biotin-GCTCGCCCCGCCCCGATCGAAT-3′ |
| Sp1 competitor | |
| Sp1CF (SEQ ID NO:37): | 5′-ATTCGATCGGGGCGGGGCGAGC-3′ |
| Sp1CR (SEQ ID NO:38): | 5′-GCTCGCCCCGCCCCGATCGAAT-3′ |

The position of polymorphic nucleotide was bolded and underlined.

Probes were synthesized as single strand and end labeled using biotin. Unlabeled oligonucleotides with the same sequences were used as competitors. Double-stranded DNA was made by the annealing of two complementary oligonucleotides. EMSA was performed using the LightShift Chemiluminescent EMSA Kit (Pierce, Rockford, USA) according to the manufacture's instructions.

Briefly, binding reactions were performed by incubating the nuclear extracts with the binding buffer (100 mM Tris-HCl, pH 7.5; 500 mM NaCl, 25 mM MgCl₂, and 5 mM dithiothreitol), 1 μg poly(dI-dC), and 0.2 pmol (200,000 cpm) labeled probe for 20 minutes at room temperature. For competition assays, 100-fold molar excess of unlabeled oligonucleotides (specific, nonspecific, or Sp1 specific) were included in the binding reaction. After binding, the samples were separated in a 5% nondenaturing polyacrylamide gel in 0.5×TBE for 2 hours at 4° C. Binding reactions were then transferred to a nylon membrane (Amersham Pharmacia Biotech) by electrophoresis in 0.5×TBE, at 100V for 40 minutes. After transfer, DNA was cross-linked at 120 mJ/cm² under 254 nm UV light. Biotin-labeled DNA was detected and visualized using the chemiluminescent based detection procedure in the ChemiDoc system (Bio-Rad).

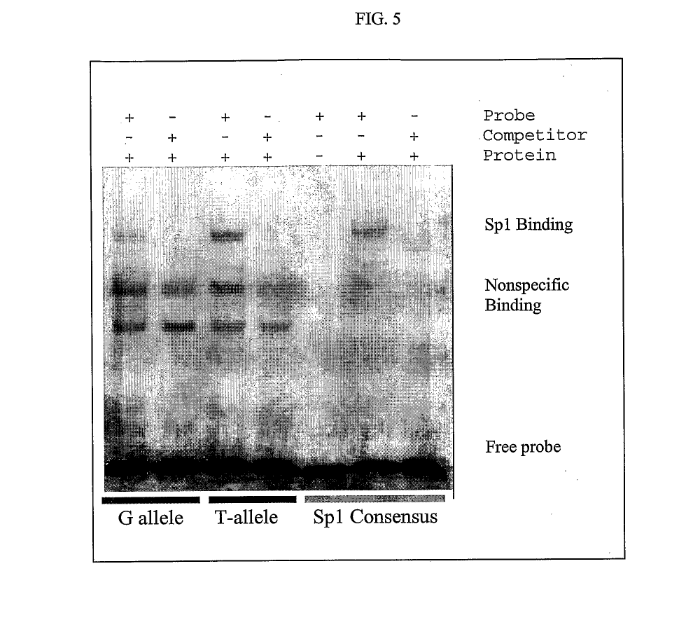

EMSA was performed to test the binding efficiency of nuclear proteins to each allele specific probe. The Sp1 consensus probe was used as the control to show the binding and position of shifting. Significantly higher binding efficiency of nuclear protein from MDA-MB-231 cells was observed with the T allele probe compared to the G allele probe (FIG. 5).

Haplotypes of −216G/T−191C/A Were Associated with EGFR mRNA Expression In Vivo.

Human fibroblast cells (which express EGFR) were selected to evaluate the association between −216G/T−191C/A haplotypes and EGFR transcription. According to the previous reports, there were multiple transcription initiation sites in the EGFR promoter (Johnson et al, 1988; Kageyama et al., 1988), while the major site for in vivo transcription was at position −260 (Kageyama et al., 1988). Thus, the positions −216 and −191 would be present in most EGFR mRNA sequences. Ten cell lines with diplotype G-C/T-C for the two polymorphisms were chosen so that there was the potential to detect a difference of expression level between mRNA carrying T-C haplotype and G-C haplotype within the same cell. As a result, a significant deviation of the average relative ratio from the hypothetical ratio 1:1 was observed (Mean of R=1.39±0.12, 95% CI 1.11-1.67, p<0.02), demonstrating that EGFR mRNA derived from the T-C haplotype was about 40% higher than that from the G-C haplotype. This finding indicates that the −216G/T variant also has a strong impact on EGFR transcription in vivo.

In addition to the allelic imbalance, the relative expression of EGFR among the above three human cell lines was evaluated by real-time PCR. Interestingly, the EGFR level among these cells were in agreement with their diplotypes with a dramatically high level of EGFR in MDA-MBA-231 cells, but about 6-fold less in HEK293 and the lowest in MCF-7 (FIG. 6B).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,582,788
U.S. Pat. No. 4,656,127
U.S. Pat. No. 4,659,774
U.S. Pat. No. 4,682,195
U.S. Pat. No. 4,683,194
U.S. Pat. No. 4,683,195
U.S. Pat. No. 4,683,202
U.S. Pat. No. 4,683,202
U.S. Pat. No. 4,683,202
U.S. Pat. No. 4,800,159
U.S. Pat. No. 4,816,571
U.S. Pat. No. 4,883,750
U.S. Pat. No. 4,946,773
U.S. Pat. No. 4,959,463
U.S. Pat. No. 5,141,813
U.S. Pat. No. 5,264,566
U.S. Pat. No. 5,279,721
U.S. Pat. No. 5,428,148
U.S. Pat. No. 5,554,744
U.S. Pat. No. 5,574,146
U.S. Pat. No. 5,602,244
U.S. Pat. No. 5,645,897
U.S. Pat. No. 5,705,629
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,849,481
U.S. Pat. No. 5,849,483
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,770
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,337
U.S. Pat. No. 5,866,366
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,925,525
U.S. Pat. No. 5,928,862
U.S. Pat. No. 5,928,869
U.S. Pat. No. 5,928,870
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 5,952,174
Akimoto et al., Clin. Cancer Res. 5:2884-2890, 1999.
Ausubel et al., In: Current Protocols in Molecular Biology, John, Wiley & Sons, Inc, New York, 1996.
Brandt et al., Cancer Res., 64:7-12, 2004.
Buerger et al., Cancer Res., 60(4):854-857, 2000.
Courey et al., Cell, 55:887-98, 1988.
Deb et al., Oncogene, 9:1341-1349, 1994.
European Appln. 201,184
European Appln. 237,362
European Appln. 258,017
European Appln. 329 822
European Appln. 50,424
European Appln. 84,796
European Patent 266,032
French Patent 2,650,840
Froehler et al., Nucleic Acids Res., 14(13):5399-5407, 1986.
Frohman, In: PCR Protocols: A Guide To Methods And Applications, Academic Press, N.Y., 1994.
Gebhardt et al., J. Biol. Chem., 274(19):13176-13180, 1999.
Grandis et al., Nature Med., 2:237-240, 1996.
Great Britain Appln. 2 202 328
Haley et al., Oncogene Res., 1(4):375-396, 1987.
Halushka et al., Nat. Genet., 22(3):239-247, 1999.
Hudson et al., Mol. Endocrinol., 3:400-408, 1989.
Inazuka et al., Genome Res, 7(11):1094-1103, 1997.
Innis et al., Proc. Natl. Acad. Sci. USA, 85(24):9436-9440, 1988.
Ishii et al., Proc. Natl. Acad. Sci. USA, 82(15):4920-4924, 1985.
Johnson et al., Nat. Genet., 29(2):233-237, 2001.
Johnson et al., J. Biol. Chem., 263(12):5693-5699, 1988.
Johnson et al., Front Biosci. 3:d447-d4488, 1998.
Kageyama et al, J. Biol. Chem., 263(13):6329-6336, 1988.
Ke and Cardon, Bioinformatics, 19(2):287-288, 2003.
Komher, et al., Nucl. Acids. Res. 17:7779-7784, 1989.
Kuppuswamy, et al., Proc. Natl. Acad. Sci. (U.S.A.) 88:1143-1147, 1991.
Kwoh et al., Proc. Natl. Acad. Sci. USA, 86:1173, 1989.
Kwok, Annu. Rev. Genomics Hum. Genet., 2:235-58, 2001.
Kwok and Chen, Curr. Issues Mol. Biol., 5(2):43-60, 2003.
Kwok et al., J. Med Genet., 33(6):465-468, 1996.
Kwok et al., Genomics, 23(1):138-144, 1994.
Landegren, et al., Science 241:1077-1080, 1988.
Maekawa et al., J. Biol. Chem., 264(10):5488-5494, 1989.
Magistroni et al., J. Nephrology, 16:110-115,2003.
Martin et al., Digestion, 66:121-126, 2002.
Maxam, et al., Proc. Natl. Acad. Sci. USA, 74:560, 1977.
Mullis et al., Cold Spring Harbor Symp. Quant. Biol. 51:263-273, 1986.
Nickerson et al., Proc. Natl. Acad. Sci. USA, 87:8923-8927, 1990.
Nyren et al., Anal. Biochem. 208:171-175, 1993.
Ohara et al., Proc. Natl. Acad. Sci. USA, 86:5673-5677, 1989.
PCT Appln WO91/02087
PCT Appln. PCT/US87/00880
PCT Appln. PCT/US89/01025
PCT Appln. WO 88/10315
PCT Appln. WO 89/06700
PCT Appln. WO 92/15712
Prezant et al., Hum. Mutat., 1:159-164, 1992.
Reinshagen et al., Gastroenterology, 104:A642, 1993.
Salomon et al., Crit. Rev. Oncol. Hematol. 19:183-232, 1995.
Sambrook et al., In: Molecular cloning, Cold Spring Harbor Laboratory Press, Cold Spring Harbor, NY, 2001.
Sanger, et al., J. Molec. Biol., 94:441, 1975.
Satsangi et al., Nat. Genet., 14:199-202, 1996.
Sokolov, Nucl. Acids Res. 18:3671, 1990.
Subler et al., Oncogene, 9:1351-1359, 1994.
Sweeney et al., Kidney Int., 55:1187-1197, 1999.
Syvanen et al., Genomics 8:684-692, 1990.
Taillon-Miller et al, Genome Res, 8(7):748-754, 1998.
Tysnes et al., Invasion Metastasis, 17:270-280, 1997.
Ugozzoll et al., GATA 9:107-112, 1992.
Walker et al., Proc. Natl. Acad. Sci. USA, 89:392-396 1992.
Wosikowski et al., Biochim. Biophys. Acta, 1497:215-226, 2000.
Xu et al. Proc. Natl. Acad. Sci. USA, 81:7308-7312, 1984.
Xu et al., J. Biol. Chem., 268:16065-16073, 1993.

1. A method for evaluating the potential efficacy of an EGFR-targeting therapeutic agent for the treatment of cancer in a patient comprising determining the sequence of a polymorphism in one or both EGFR genes in the patient.

2. The method of claim 1, wherein the polymorphism is at, or in linkage disequilibrium with, a nucleotide position selected from the group consisting of nucleotide positions −1435, −1300, −1249, −1227, −761, −650, −544, −486, −216, −191, 169, and 2034.

3. The method of claim 2, wherein the polymorphism is, or is in linkage disequilibrium with, a polymorphism selected from the consisting of −1435 C>T, −1300 G>A, −1249 G>A, −1227 G>A, −761 C>A, −650 G>A, −544 G>A, −486 C>A, −216 G>T, −191 C>A, 169 G>T, and 2034 G>A.

4. The method of claim 1, further comprising determining the sequence of at least two polymorphisms in one or both EGFR genes in the patient.

5. The method of claim 1, wherein the EGFR-targeting therapeutic agent is an EGFR-tyrosine kinase inhibitor.

6. The method of claim 5, wherein the EGFR-tyrosine kinase inhibitor is gefitinib or erlotinib.

7. The method of claim 1, wherein the EGFR-targeting therapeutic agent is a monoclonal antibody.

8. The method of claim 7, wherein the monoclonal antibody is cetuximab.

9. (canceled)

10. The method of claim 8, wherein a T at position −216 on an allele is an indicator of higher expression of EGFR protein, and further wherein the higher expression of EGFR protein is an indicator of decreased efficacy of the EGFR-targeting therapeutic agent.

11. The method of claim 1, further comprising determining the sequence of a polymorphism in both EGFR genes in the patient.

12. The method of claim 1, wherein determining the sequence of a polymorphism is performed by a hybridization assay, by an allele specific amplification assay, by a sequencing or a microsequencing assay, or by digestion with a restriction enzyme.

13-15. (canceled)

16. The method of claim 1, further comprising obtaining a sample.

17. The method of claim 16, wherein the sample comprises buccal cells, mononuclear cells, or cancer cells.

18. The method of claim 1, further comprising administering the EGFR-targeting therapeutic agent to the patient.

19. A method for predicting the clinical prognosis for a cancer patient comprising determining the sequence of a polymorphism in one or both EGFR genes in the patient.

20. The method of claim 19, further comprising determining the sequence of a polymorphism in both EGFR genes in the patient.

21. The method of claim 19, wherein the polymorphism is at, or in linkage disequilibrium with, a nucleotide position selected from the group consisting of nucleotide positions −1435, −1300, −1249, −1227, −761, −650, −544, −486, −216, −191, 169, and 2034.

22. The method of claim 21, wherein the polymorphism is, or is in linkage disequilibrium with, a polymorphism selected from the consisting of −1435 C>T, −1300 G>A, −1249 G>A, −1227 G>A, −761 C>A, −650 G>A, −544 G>A, −486 C>A, −216 G>T, −191 C>A, 169 G>T, and 2034 G>A.

23. (canceled)

24. The method of claim 19, wherein a T at position −216 on an allele is an indicator of an increased expression of EGFR protein.

25. The method of claim 24, wherein the increased expression of EGFR protein is predictive of poor prognosis.

26. The method of claim 25, wherein the poor prognosis indicates increased resistance to chemotherapy, hormonal therapy, or radiotherapy.

27. The method of claim 25, wherein the poor prognosis indicates increased risk of metastasis.

28. A method for evaluating a patient's risk of toxicity to an EGFR-targeting therapeutic agent comprising determining the sequence of a polymorphism in one or both EGFR genes in the patient.

29. The method of claim 28, wherein the polymorphism is at, or in linkage disequilibrium with, a nucleotide position selected from the group consisting of nucleotide positions −1435, −1300, −1249, −1227, −761, −650, −544, −486, −216, −191, 169, and 2034.

30. The method of claim 29, wherein the polymorphism is, or is in linkage disequilibrium with, a polymorphism selected from the consisting of −1435 C>T, −1300 G>A, −1249 G>A, −1227 G>A, −761 C>A, −650 G>A, −544 G>A, −486 C>A, −216 G>T, −191 C>A, 169 G>T, and 2034 G>A.

31. (canceled)

32. The method of claim 30, wherein a T at position −216 on one or both alleles is an indicator of decreased toxicity of the EGFR-targeting therapeutic agent.

33. The method of claim 28, further comprising determining the sequence of a polymorphism in both EGFR genes in the patient.

34-35. (canceled)

36. A kit for evaluating the potential efficacy of an EGFR-targeting therapeutic agent in a patient comprising a nucleic acid for determining the sequence of a polymorphism in an EGFR gene locus or a restriction enzyme for determining the sequence of a polymorphism in an EGFR gene locus.

37. The kit of claim 36, wherein the nucleic acid is a primer for amplifying a polymorphism at a nucleotide position selected from the group consisting of −1435, −1300, −1249, −1227, −761, −650, −544, −486, −216, −191, 169, and 2034.

38. The kit of claim 36, wherein the nucleic acid is a specific hybridization probe designed to detect a polymorphism at a nucleotide position selected from the group consisting of −1435, −1300, −1249, −1227, −761, −650, −544, −486, −216, −191, 169, and 2034.

39. The kit of claim 38, wherein the specific hybridization probe is comprised in an oligonucleotide array or microarray.

40. (canceled)


SEQUENCE LISTING (38 sequences)

SEQ ID NO: 1 - DNA, 525 bp, Homo sapiens
gaaattaactcctcagggcacccgctcccctcccatgcgccgccccactcccgccggagactaggtcccgcgggggccaccgctgtccaccgcctccggcggccgctggccttgggtccccgctgctggttctcctccctcctcctcgcattctcctcctcctctgctcctcccgatccctcctccgccgcctggtccctcctcctcccgccctgcctccccgcgcctcggcccgcgcgagctagacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggcc

SEQ ID NO: 2 - DNA, 4990 bp, Homo sapiens
ctccacagaggctgtgagctagagccctaactgtgcagggccctaactatgccaggctacttatctctcttaagaggacttcattagtgcctgctcggccatacagttttttacttaccaagtaacacagttatcagcacactccaggtactagccaaggactacaaaatcaacgtgaatgtcagcttttgtatcaaaagctcaaaggagaaactcaaactttacatagatgtcccatgaagatgttcagcaaacccattcttctctgttccctggaatccatcccagtattgtgctatgtgtgtgtctagtaattctttacaaaaagctctgtttcttgtgatgctatcagatcacattgaagaatatacaagccgtactatgaaggctgttgtctcatatagtcctaacgtagtgagaactgatgttcttacatgctgtctttttgggcactcaaagaaattcctgtacagtcttacaaatcagttgtagcttaaattgatttgtgttgtgacttgtacacacaggtcacattcccttgacagaaaatatagtttaaaaccaaatttgcagcccttgttaagtgaatgcacaggactttattgtattcaggtcttttattgtaagactcactcctgtcttcattttatgttccactgttgtgcttcccatttgcctttctctagttttgttttctgtgtttctacggactgctctcagcccaggtgtgcaggaagcacacacatgcctgcagagccttcatggcctctgcattcagggcatgacttcaacgcacagtggctgtactgatttgttaaaacaaaggaacagattacttctcctaattcacagggaagttccaggttgtgcgggcagtgagcagacctgtgtctgtctgcgcttgccctggtgaaaaaccccaccgttcaggctgcagggtgcgagacccaggcacaaacattttgctggatgaggaggaaagatgtaaggttgctccccttcagagacagcaaagggcaggtctgtagcttcacttacttcaggattgtgatttttgacagagccgagagatcagggttgttgaaccaggcctgaaggtcctagtgaatctcgtgaagagaggaggggtctggctgtaacatggacctagaggacatttttactgcaggagaaggaacagtggggatggggtggacttgccaaaggaatatagctcaagttcctgcagcccaaaaaagctcagtttcttttggccaaagcttccgcgagtttccctggcatttctcctgcgggagctacaggggcagtgggacacttagcctctctaaaagcacctccacggctgtttgtgtcaagcctttattccaagagcttcacttttgcgaagtaatgtgcttcacacattggcttcaaagtacccatggctggttgcaataaacattaaggaggcctgtctctgcacccggagttgggtgccctcatttcagatgatttcgagggtgcttgacaagatctgaaggaccctcggactttagagcaccacctcggacgcctggcacccctgccgcgcgggcacggcgacctcctcagctgccaggccagcctctgatccccgagagggtcccgtagtgctgcaggggaggtggggacccgaataaaggagcagtttccccgtcggtgccattatccgacgctggctctaaggctcggccagtctgtctaaagctggtacaagtttgctttgtaaaacaaaagaagggaaagggggaaggggaccctggcacagatttggctcgacctggacataggctgggcctgcaagtccgcggggaccgggtccagaggggcagtgctgggaacgcccctctcggaaattaactcctcagggcacccgctcccctcccatgcgccgccccactcccgccggagactaggtcccgcgggggccaccgctgtccaccgcctccggcggccgctggccttgggtccccgctgctggttctcctccctcctcctcgcattctcctcctcctctgctcctcccgatccctcctccgccgcctggtccctcctcctcccgccctgcctccccgcgcctcggcccgcgcgagctagacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaaggtaagggcgtgtctcgccggctcccgcgccgcccccggatcgcgccccggaccccgcagcccgcccaaccgcgcaccggcgcaccggctcggcgcccgcgcccccgcccgtcctttcctgtttccttgagatcagctgcgccgccgaccgggaccgcgggaggaacgggacgtttcgttcttcggccgggagagtctggggcgggcggaggaggagacgcgtgggacaccgggctgcaggccaggcggggaacggccgccgggacctccggcgccccgaaccgctcccaactttcttccctcactttccccgcccagctgcgcaggatcggcgtcagtgggcgaaagccgggtgctggtgggcgcctggggccggggtcccgcacgtgcgccccgcgctgtcttcccagggcgcgacggggtcctggcgcgcacccgaggggcgggcgctgcccacccgccgagactgcactgtttagggaagctgaggaaggaacccaaaaatacagcctcccctcggaccccgcgggacaggcggctttctgagaggacctccccgcctccgccctccgcgcaggtctcaaactgaagccggcgcccgccagcctggccccggcccctctccaggtccccgcgatcctcgttccccagtgtggagtcgcagcctcgacctgggagctgggagaactcgtctaccaccacctgcggctcccggggaggggtggtgctggcggcggttagtttcctcgttggcaaaaggcaggtggggtccgacccgccccttgggcgcagaccccggccgctcgcctcgcccggtgcgccctcgtcttgcctatccaagagtgccccccacctcccggggaccccagctccctcctgggcgcccgcgccgaaagccccaggctctccttcgatggccgcctcgcggagacgtccgggtctgctccacctgcagcccttcggtcgcgcctgggcttcgcggtggagcgggacgcggctgtccggccactgcagggggggatcgcgggactcttgagcggaagccccggaagcagagctcatcctggccaacaccatggtgtttcaaaatggggctcacagcaaacttctcctcaaaacccggagactttctttcttggatgtctctttttgctgtttgaagaatttgagccaaccaaaatattaaacctgtcttacacacacacacacacacacacacacacacacaccggattgctgtccctggttcaagtgtgccaagtgtgcagacagaacatgagcgagtctggcttcgtgactaccgaccataaacccacttgacaggggaaacatgccttggaaggtttaattgcacaattccaaccttgagctgcgcgggttccaagagccaggcccgtacttgctgttgatgtcattggcttggggagttggggtttggtgcccagcgcggtcgttgggggaggggcaaggcatagaacagtggttcccagaccttgctgcacattggaattacctgggattaaaaaaaaaaaaatcaaaacaaaaaccagtgtctggctcccgcccccagacattctgatttaattggcatggggcaagacctggacttgggattttttttaatgctcttcatgtgatctgttgggcagccagatttggggatcactagacggaagaaggattgttaaagtctccggagatgttacttgccaatgctaagagctctttgaggacatctggaattgttacaatattgccaaatataggaaagagggaaaaggtagagtgtgattccaataataaaggattccgcttttcattgaaggaactggtggaaaggtttcttctctgctgagcctgcaggcccgtcctgcctgcctggggtgcccgggagacgcgggcctgctccggagactgctgactgccggtcctgttagtcaggtgtcagccctgtctctgccgaagagactcttctctttattttaaattaaaccctcagagcaccaccaaagcatcacttttctccctccattggtgttctcattctttgatgttacttgtttgaacaccactattagtagttggagatttgttcctgagaaaaatataaataccacttaatttgcctgtttgtcccgcattcactcaaaacagaatgctcctgaagacaagagagagagtaggagaacagacgctattccattacagtaacataaaagactggattttcaggggcaaattattaaaataggagatgagctcttttaacagaaatttgtttaaggcctgtgtctatcaaattcagtggattttattcaagatgcactttgtttagtgggagttttgtttggttctgggacatgctaacttctagacttgctgctcttagaggtaatgactgccagacaccatttcatgagtcctaatccccacattaagcataagaggtgcacactctcctcctatgggggaaactgaggtacgaagaactaaagtgactttcccacagctggtgggaggcagacggga

SEQ ID NO: 3 - DNA, 20 bp, Artificial Sequence
  Description of Artificial Sequence Synthetic Primer
gttccactgttgtgcttccc

SEQ ID NO: 4 - DNA, 20 bp, Artificial Sequence
  Description of Artificial Sequence Synthetic Primer
aagaaagttgggagcggttc

SEQ ID NO: 5 - DNA, 19 bp, Artificial Sequence
  Description of Artificial Sequence Synthetic Primer
gggtggacttgccaaagga

SEQ ID NO: 6 - DNA, 20 bp, Artificial Sequence
  Description of Artificial Sequence Synthetic Primer
cttagagccagcgtcggata

SEQ ID NO: 7 - DNA, 20 bp, Artificial Sequence
  Description of Artificial Sequence Synthetic Primer
gcatgacttcaacgcacagt

SEQ ID NO: 8 - DNA, 20 bp, Artificial Sequence
  Description of Artificial Sequence Synthetic Primer
gaggctaagtgtcccactgc

SEQ ID NO: 9 - DNA, 20 bp, Artificial Sequence
  Description of Artificial Sequence Synthetic Primer
tcggactttagagcaccacc

SEQ ID NO: 10 - DNA, 20 bp, Artificial Sequence
  Description of Artificial Sequence Synthetic Primer
gaggaggagaatgcgaggag

SEQ ID NO: 11 - DNA, 21 bp, Artificial Sequence
  Description of Artificial Sequence Synthetic Primer
aaattaactcctcagggcacc

SEQ ID NO: 12 - DNA, 20 bp, Artificial Sequence
  Description of Artificial Sequence Synthetic Primer
cgcccttacctttcttttcc

SEQ ID NO: 13 - DNA, 20 bp, Artificial Sequence
  Description of Artificial Sequence Synthetic Primer
ccctgactccgtccagtatt

SEQ ID NO: 14 - DNA, 20 bp, Artificial Sequence
  Description of Artificial Sequence Synthetic Primer
cgtcctttcctgtttccttg

SEQ ID NO: 15 - DNA, 20 bp, Artificial Sequence
  Description of Artificial Sequence Synthetic Primer
accagctgtgggaaagtcac

SEQ ID NO: 16 - DNA, 20 bp, Artificial Sequence
  Description of Artificial Sequence Synthetic Primer
agacgagttctcccagctcc

SEQ ID NO: 17 - DNA, 20 bp, Artificial Sequence
  Description of Artificial Sequence Synthetic Primer
gcgcaggtctcaaactgaag

SEQ ID NO: 18 - DNA, 20 bp, Artificial Sequence
  Description of Artificial Sequence Synthetic Primer
ggagaagtttgctgtgagcc

SEQ ID NO: 19 - DNA, 19 bp, Artificial Sequence
  Description of Artificial Sequence Synthetic Primer
ccctcgtcttgcctatcca

SEQ ID NO: 20 - DNA, 20 bp, Artificial Sequence
  Description of Artificial Sequence Synthetic Primer
agtgatccccaaatctggct

SEQ ID NO: 21 - DNA, 20 bp, Artificial Sequence
  Description of Artificial Sequence Synthetic Primer
ggcatagaacagtggttccc

SEQ ID NO: 22 - DNA, 20 bp, Artificial Sequence
  Description of Artificial Sequence Synthetic Primer
gaacaccaatggagggagaa

SEQ ID NO: 23 - DNA, 20 bp, Artificial Sequence
  Description of Artificial Sequence Synthetic Primer
tgaaggaactggtggaaagg

SEQ ID NO: 24 - DNA, 20 bp, Artificial Sequence
  Description of Artificial Sequence Synthetic Primer
catgtcccagaaccaaacaa

SEQ ID NO: 25 - DNA, 28 bp, Artificial Sequence
  Description of Artificial Sequence Synthetic Primer
ccaccggtaccggcggccgctggccttg

SEQ ID NO: 26 - DNA, 23 bp, Artificial Sequence
  Description of Artificial Sequence Synthetic Primer
cggcgagacacgcccttaccttt

SEQ ID NO: 27 - DNA, 25 bp, Artificial Sequence
  Description of Artificial Sequence Synthetic Primer
gcagcctccgccccccgcacggtgt

SEQ ID NO: 28 - DNA, 25 bp, Artificial Sequence
  Description of Artificial Sequence Synthetic Primer
acaccgtgcggggggcggaggctgc

SEQ ID NO: 29 - DNA, 25 bp, Artificial Sequence
  Description of Artificial Sequence Synthetic Primer
gcagcctccgccccccgcacggtgt

SEQ ID NO: 30 - DNA, 25 bp, Artificial Sequence
  Description of Artificial Sequence Synthetic Primer
acaccgtgcggggggcggaggctgc

SEQ ID NO: 31 - DNA, 25 bp, Artificial Sequence
  Description of Artificial Sequence Synthetic Primer
gcagcctcctccccccgcacggtgt

SEQ ID NO: 32 - DNA, 25 bp, Artificial Sequence
  Description of Artificial Sequence Synthetic Primer
acaccgtgcggggggaggaggctgc

SEQ ID NO: 33 - DNA, 25 bp, Artificial Sequence
  Description of Artificial Sequence Synthetic Primer
gcagcctcctccccccgcacggtgt

SEQ ID NO: 34 - DNA, 25 bp, Artificial Sequence
  Description of Artificial Sequence Synthetic Primer
acaccgtgcggggggaggaggctgc

SEQ ID NO: 35 - DNA, 22 bp, Artificial Sequence
  Description of Artificial Sequence Synthetic Primer
attcgatcggggcggggcgagc

SEQ ID NO: 36 - DNA, 22 bp, Artificial Sequence
  Description of Artificial Sequence Synthetic Primer
gctcgccccgccccgatcgaat

SEQ ID NO: 37 - DNA, 22 bp, Artificial Sequence
  Description of Artificial Sequence Synthetic Primer
attcgatcggggcggggcgagc

SEQ ID NO: 38 - DNA, 22 bp, Artificial Sequence
  Description of Artificial Sequence Synthetic Primer
gctcgccccgccccgatcgaat